(19) United States
(12) Patent Application Publication
(10) Pub. No.: US 2012/0136189 A1
(43) Pub. Date: May 31, 2012

(54) BUFFERED IONIC LIQUIDS FOR OLEFIN DIMERIZATION

(75) Inventors: Matthias Dötterl, Selbitz (DE); Helmut G. Alt, Bayreuth (DE); Roland Schmidt, Bartlesville, OK (US)

(73) Assignee: CONOCOPHILLIPS COMPANY - IP SERVICES GROUP, Houston, TX (US)

(21) Appl. No.: 13/025,935

(22) Filed: Feb. 11, 2011

Related U.S. Application Data
(60) Provisional application No. 61/312,142, filed on Mar. 9, 2010.

(51) Int. Cl. C07C 2/24

(52) U.S. Cl. 585/513

(57) ABSTRACT

The present invention relates generally to buffered ionic liquids that are very useful for dimerization of olefins, such as isopropene, wherein the buffer is a phosphine or a bismuthine or an arsine or an amine.

### CROSS REFERENCE TO RELATED APPLICATIONS

This application claims priority to U.S. Ser. No. 61/312,142, filed Mar. 9, 2010 and is incorporated by reference herein.

### FEDERALLY SPONSORED RESEARCH STATEMENT

Not applicable.

### Reference to Microfiche Appendix

Not applicable.

### FIELD OF THE INVENTION

The present invention relates generally to buffered ionic liquids, particularly to buffered ionic liquids that can be used to oligomerize olefins. The buffer can be selected from the group consisting of aryl and phenyl compounds of Bi, P, N, As, and Sb, wherein the buffer can contribute to dimer selectivity.

### BACKGROUND OF THE INVENTION

Dimerization of olefins is well known and industrially useful. In particular, dimerization of 2-methylpropene to produce 2,4,4-trimethylpentene, commonly called isooctane, is a well-known and useful reaction, because the product can be used for gasoline reformulation. Branched saturated hydrocarbons, such as isooctane, have a high octane number, low volatility and do not contain sulfur or aromatics, and are, therefore, particularly useful for improving gasoline and making it more environmentally friendly. Dimerizing linear olefins also represents an attractive route for producing high octane number blending components. In general, the branched species have higher octane value, although they may also contribute to engine deposits. Thus, in some instances the lower octane number of products of dimerization of linear olefins may be offset by lower engine deposits.

Branched saturated hydrocarbons can be produced in different ways, e.g. by alkylation of olefins with isoparaffins and by dimerization of light olefins, in some instances followed by hydrogenation. Alkylation of 2-methylpropene (isobutene) with isobutane directly produces isooctane, and the dimerization reaction of 2-methylpropene produces 2,4,4-trimethyl-1-pentene and 2,4,4-trimethyl-2-pentene, amongst other products. FIGS. 1A and 1B illustrate such alkylation/dimerization and the products thereof. These eight carbon species can be used in gasoline, provided the alkene limitations of gasoline are not exceeded. If use results in exceeding alkene limitations of a gasoline, such alkenes can be converted into alkanes by hydrogenation prior to use in gasoline.

Use of ionic liquids for dimerization (and oligomerization) of olefins is also well-known. In the broad sense, the term ionic liquid (IL) includes all molten salts, for instance, sodium chloride at temperatures higher than 800° C. Currently, the term “ionic liquid” is commonly used for salts whose melting point is relatively low (below about 100° C.).

Ionic liquids make an ideal solvent because they have very low volatility, and do not evaporate or burn easily, resulting in safer processes. Also, the low melting point and negligible vapor pressure lead to a wide liquid range often exceeding 100° C., unlike water which vaporizes at 100° C. Another advantage is that chemical and physical properties of ionic liquids can be “tuned” by selecting different anion and cation combinations, and different ionic liquids can be mixed together to make binary or ternary ionic liquids. Ionic liquid solvents can also function as catalysts or cocatalysts in reactions.

Using ionic liquids in oligomerization reactions simplifies product separation. Most ionic liquids are polar, and hence non-polar products—like isooctane and octane—are immiscible therein. The biphasic process allows separation of the products by decantation and easy recycling of the catalysts. Further, the fact that the product is not miscible in the solvent, tends to drive the reaction towards dimer production, rather than less useful trimers and tetramers. Thus, the selectivity of the reaction for dimer formation is greatly increased.

Several groups have shown that a wide range of acidic chloroaluminate(III) and alkylchloroaluminate(III) ionic liquids catalyzed cationic oligomerizations of alkenes. Ionic liquid included 1-alkyl-3-methylimidazolium chloride/AlCl₃, x(AlCl₃)>0.5, butylpyridinium chloride/AlCl₃ (1:2), hydrogenpyridinium chloride/AlCl₃ (1:2), [C₄mim]Cl/AlCl₃/EtAlCl₂ (1:1.1:0.1) and imidazolium chloride/AlCl₃ (2:3). The reactions were not very selective, as dimers and also odd-numbered hydrocarbons were produced, but using an ionic liquid in the polymerization process made product separation easy.

The Institut Francaise du Petrole (IFP) has developed a monophasic process for the dimerization of alkenes that is known as the DIMERSOL™ process. The Dimersol™ process is operated in the liquid phase without a solvent at temperatures between 40-60° C. and at a pressure of 18 bars with a cationic nickel complex [PR₃NiCH₂R′]⁺[AlCl₄]⁻. In the Dimersol™ X process the conversion of butenes is 80% and the selectivity toward octenes is 85%. The process has a low capital cost, as it is operated at low temperatures and at low pressure, but product separation from the catalyst is a major problem. Also, the catalyst is not recycled, thus increasing operational costs.

IFP has since modified its Dimersol™, process so that it uses a BMIM/Cl/AlCl₃/EtAlCl₂ (1:1.2:0.1) ionic liquid in the dimerization reactions (see e.g., WO2007080287). The process is called DIFASOL™ and its biphasic nature allows easier product separation and catalyst recycling. The same cationic nickel complex [PR₃NiCH₂R′]⁺ [AlCl₄]⁻ is applied as a catalyst, but being polar it does not partition into the apolar product phase, and thus it is easily recycled with the ionic liquid. As a result, nickel consumption is decreased by a factor of 10. The conversion of butene is 80-85% and dimer selectivity is increased to 90-95%.

Wasserscheid and Keim (WO9847616) developed an alternative alkylaluminum-free IL for the dimerization of 1-butene to producing linear dimers. Alkylaluminum dichloride is known to exhibit strong isomerization activity. Instead, weak organic bases (such as pyrrole, pyridine, quinoline and derivatives thereof) were applied to reduce the acidity of the Al₂Cl₇⁻ species in the ionic liquid that could catalyze the non-selective, cationic oligomerization reaction. The base, therefore, should have the following properties: 1) sufficient reactivity to eliminate all free acidic species in the IL; 2) non-coordinating with respect to the catalytic active Ni center; 3) high solubility in the ionic liquid and not partition into the organic product layer; and 4) inert against the butene or other feedstock and the oligomerization products. Hence, a possible base would be any cyclic, heterocyclic, or aliphatic, aromatic or non-aromatic base. The results of one study of several nitrogen bases are excerpted below:

TABLE A
Effect of the base on product distribution in the dimerization
reaction of 1-butene in a [C₄mim]Cl/AlCl₃/base ionic liquid
catalyzed with nickel complex (cod)Ni(hfacac).

| | | | Linear |
|---|---|---|---|
| Base | TOF h⁻¹ | Dimers % | dimers % |
| Pyrrole | 1350 | 86 | 56 |
| N-methylpyrrole | 2100 | 98 | 51 |
| Chinoline | 1240 | 98 | 64 |
| Pyridine | 550 | 78 | 33 |
| 2,6-Lutidine | 2480 | 55 | 68 |
| Di-tert-butylpyridine | 2100 | 49 | 68 |
| 2,6-Dichloropyridine | 56 | 34 | 74 |
| 2,6-Difluoropyridine | 730 | 29 | 72 |

TOF = Turnover frequency in mol of butene converted per mol of nickel per hour.

Although all of the above methods are known and used in the synthesis of olefin dimers and oligomers, what is needed in the art is an improved synthetic method that allows for easy separation of the product, maximum reuse of ingredients, and results in almost complete conversion of monomers to dimers with very high selectivity.

### BRIEF DESCRIPTION OF THE DRAWINGS

FIG. 1A shows the dimerization of 2-methylpropene and FIG. 1B shows a full range of isomers that might be produced in the dimerization of 2-methylpropene. 2-methylpropene 7; 2,4,4-trimethyl-1-pentene 8 (bp. 101.4° C.); 2,4,4-trimethyl-2-pentene 9 (bp. 104.9° C.); 2,3,4-trimethyl-1-pentene 33 (bp. 108° C.); 2,3,4-trimethyl-2-pentene 34 (bp. 116.5° C.); 2,3,3-trimethyl-1-pentene 37 (bp. 108.3° C.); 3,3,4-trimethyl-1-pentene 35 (bp. 105° C.); 3,4,4-trimethyl-2-pentene 36 (bp. 112° C.); and 3,4,4-trimethyl-1-pentene 66 (bp. 104° C.). Trimers and higher oligomers can also be formed (not shown in FIGS. 1A and 1B).

FIG. 2. Catalyst useful in the processes described herein.

FIG. 3 illustrates the cations used in the runs described in Table 10.

FIG. 4 illustrates the cations used in the runs described in Table 11.

FIG. 5 illustrates the cations used in the runs described in Table 12.

FIG. 6 illustrates a possible recycle scheme for a propene dimerizing ionic liquid system based on non-polar aliphatic hydrochloride salts of tertiary amines.

FIG. 7 illustrates the cations used in the runs described in Table 13.

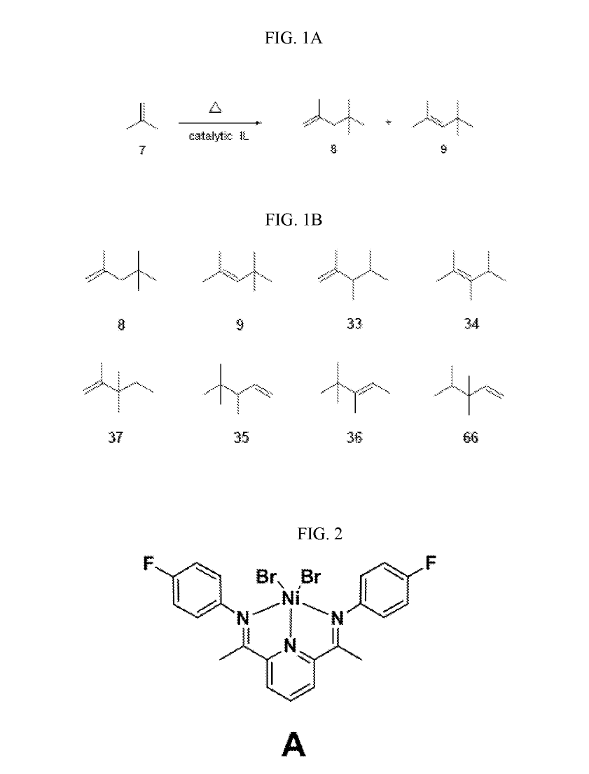

### DESCRIPTION OF EMBODIMENTS OF THE INVENTION

This application uses the following abbreviations:

| ABBREVIATION | NAME |
|---|---|
| (COD)Ni(HFACAC) | Ni-cyclooctadienyl hexafluoroacetyl |
| | acetonate |
| AlCl₃ | Aluminum trichloride or Trichloroalumnium |
| AsPh₃ | Trisphenylarsine |
| BMIM | Butylmethylimidazolium |
| BIF₃ | Bismuth(III) fluoride |
| BII₃ | Bismuth(III) iodide |
| BIPH₃ | Triphenylbismuthane or Triphenylbismuthine |
| | or Triphneyl bismuth |
| BMIMCl or C₄MIMCl | 1-Butyl-3-mehtylimidazolium chloride |
| ClPPh₂ | Chlorodiphenylphosphine |
| EtAlCl₂ | Ethylaluminiumdichloride or |
| | dichlooroethylaluminum |
| HDPE | High density polyethylene |
| HNPh₂ | Diphenylamine |
| mim | Methylimidazolium |
| NPh₃ | Triphenylamine |
| P(C₆F₅)₃ | Tris(pentafluorophenyl)phosphine |
| P(m-ClC₆H₄)₃ | Tris(3-chlorophenyl)phosphine |
| P(OMe)₃ | Trimethyl phosphite |
| P(O—MeC₆H₄)₃ | Tri(o-toyl)phosphine |
| P(OPh)₃ | Triphenyl phosphite |
| P(p-BrC₆H₄)₃ | Tris(4-bromophenyl)phosphine |
| P(p-ClC₆H₄)₃ | Tris(4-chlorophenyl)phosphine |
| P(p-FC₆H₄)₃ | Tris(4-fluorophenyl)phosphine |
| P(p-MeC₆H₄)₃ | Tri(p-tolyl)phosphine |
| Ph₂P(m-NaSO₃C₆H₄) | Diphenylphosphinobenzene-3-sulfonic acid |
| | sodium salt |
| Ph₂P(p-BF₄⁻Me₃N⁺C₆H₄) | N,N,N-Trimethyl-4- |
| | diphenylphosphinoanilinium |
| | tetrafluoroborate |
| Ph₂P(p-I⁻Me₃N⁺C₆H₄) | N,N,N-Trimethyl-4- |
| | diphenylphosphinoanilinium iodide |
| Ph₂P(P—MeOC₆H₄) | Diphenyl(4-methyoxyphenyl)phosphine |
| Ph₂P-BMIMCl | 2-Diphenylphosphino-1-butyl-3- |
| | methylimidazolium chloride |
| Ph₂PFc | 1-Diphenylphosphino ferrocene |
| Ph₂POMe | Methyl diphenylphosphinite |
| Ph₂POPh | Phenyl diphenylphosphinite |
| PPh₃ | Triphenyl phosphine |
| PR₃ | Trialkylphosphine |
| SbPh₃ | Triphenylstilbine or Triphenyantimony |
| ZrCl₄ | Zirconium tetrachloride |

The inventors herein have studied various buffers to use in place of the nitrogenous bases of Wasserscheid and Keim to improve the catalytic activity and selectivity and improve the economics of the reaction. Surprisingly, aryl and phenyl compounds of Bi, P, N, As, and Sb, have been discovered to have superior properties in this regard.

The inventors have discovered that acidic ionic liquids can be buffered with phosphines (e.g. triphenylphosphine) in comparable molar ratios to the nitrogen bases as described in the Wasserscheid patent (WO9847616). This activity is similar to the one reported for the chloroalkylaluminum buffered system used in the DIFASOL™ process. However, in contrast to the DIFASOL™ process, the dimer selectivities achieved were significantly greater (approximately 90%). A summary of the advantages of the invention are presented in Table B:

TABLE B
Comparison with the Prior Art

| Properties of the | Commercial | |
|---|---|---|
| Catalytic Ionic | DIFASOL ® | |
| Liquid Systems | System (IFP) | Inventive System |
| Price for 1 kg | ~220 [symbol] | ~80 [symbol] |
| Liquid (Lab Scale) | | |
| Activity (Propene | extremely high | extremely high |
| Dimerization) | | |
| Dimer Selectivity | ~80% | >90% (up to 98%) |
| Compositions | defined compositions | almost any composition |
| | dialkylimidazolium | with excess AlCl₃ |
| | cations | many cheap cations |
| Repeatability | almost indefinite | almost indefinite |
| Reaction Type | biphasic (liquid liquid) | biphasic or heterogeneous |
| | | (silica supported) |
| Catalyst | any nickel complex | any nickel complex |
| Recycling | difficult | very easy |
| Additive Effects on | yes | yes |
| Branching | | |
| Dimerization of | yes | yes |
| other 1-Olefins | | |
| Sensitivity | extremely vs. water | extremely vs. water |
| | extremely vs. oxygen | stable vs. oxygen |
| | | not very sensitive vs. |
| | | impurities |

1-Butyl-3-methylimidazoliumchloride (BMIMCl):AlCl₃:PPh₃ in a ratio of 1:1.2:0.09-0.12 and nickel catalyst concentrations of about 0.01 mmol/ml in the ionic liquid was tested and demonstrated improved dimerization without the addition of aluminumalkyls.

The buffers of the invention include phosphines, amines and other compounds of the following formulas: PPh₃, Tri(p-tolyl)phosphine; Tri(o-tolyl)phosphine, ClPPh₂, NPh₃, HNPh₂, P(OMe)₃, P(OPh)₃, Ph₂POPh, AsPh₃, and SbPh₃.

It was also found that it is possible to buffer acidic ionic liquids with bismuthines (e.g. triphenylbismuth) in similar molar ratios to the nitrogen bases as described in the Wasserscheid patent (WO9847616). The activity was only slightly lower than the one reported for the chloroalkylaluminum buffered system used in the DIFASOL™ process, but, in contrast to the DIFASOL™ process, dimer selectivities of up to 96% were obtained. Thus, dimer selectivity was greatly improved with this buffer.

BMIMCl:AlCl₃:BiPh₃ in a ratio of 1:1.2:0.07-0.30 and nickel catalyst concentrations of approximately 0.01 mmol/ml in the ionic liquid was tested and was found to give good dimerization without the addition of aluminumalkyls. The system works over a wide range of BiPh₃ concentrations unlike the PPh₃ system, which only works between about 0.09 and 0.12 molar equivalents. Even without additional aluminumalkyls as in the case of PPh₃ or steadily supplying BiPh₃, a stable system was obtained which could be used repeatedly without significant loss of activity.

Bismuthines of the invention include those of Formula II: BiRₓR′_{Y} where x+y is 3 and R, R′ are alkyl, aryl, H, alkenyl, or alkynyl.

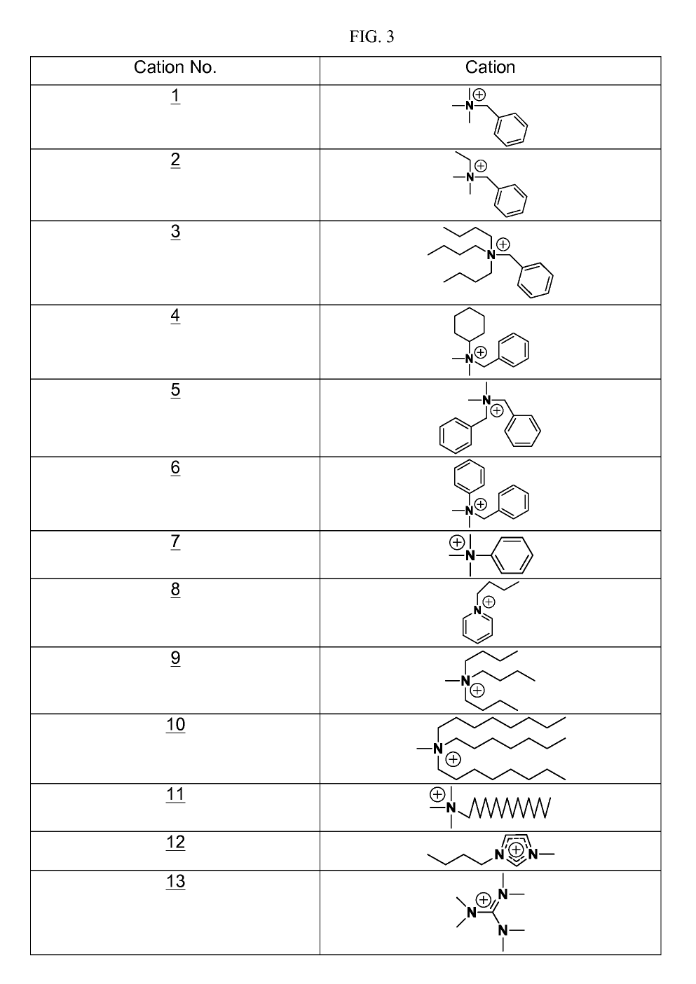

The nickel catalyst used in both the phosphine and the bismuthine experiments is shown in FIG. 2. Organometallic catalysts suitable for oligomerization that work in the chloroalkylaluminum or nitrogen base buffered system should work in the buffered systems of the present invention.

Generally speaking, embodiments of the invention include new buffers for use with acidic ionic liquid solutions employed in the oligomerization of olefins.

In one embodiment of the invention, a new form of buffered ionic liquid comprising acidic ionic liquids buffered by a phosphine buffer, such as triphenylphosphine (PPh₃) or diphenylphosphinoferrocene and derivatives thereof, is provided.

In another embodiment of the invention, a new form of buffered ionic liquids comprising an acidic ionic liquid buffered by bismuthines, such as triarylbismuthines or aromatic bismuth heterocycles are described.

In other embodiments of the invention, a new form of buffered ionic liquids comprising an acidic ionic liquid buffered by other compounds including NPh₃, HNPh₂, P(OMe)₃, P(OPh)₃, Ph₂POPh, AsPh₃, and SbPh₃ are described.

A number of unmodified and modified ionic liquids are described in WO9847616 that may be useful in the present invention. For example, ionic liquids useful herein include mixtures of salts which melt below room temperature. Such salt mixtures include aluminum halides in combination with one or more of ammonium halides, imidazolium halides, pyridinium halides, sulfonium halides and phosphonium halides, the latter being preferably substituted, for example, by alkyl groups. Examples of the substituted derivatives of the latter include one or more of 1-methyl-3-butyl imidazolium halide, 1-butyl pyridinium halide and tetrabutyl phosphonium halides. Other ionic liquids consist of a mixture where the mole ratio of AlX₃/RX (in which X represents an alkyl group, a halide or a combination thereof and R is an alkyl group) is (usually)>1.

In particular, there is provided a buffered ionic liquid comprising: a compound of the formula RₙMX₃₋ₙ or of the formula RₘM₂X₆₋ₘ, wherein (i) M is a metal selected from the group consisting of aluminum, gallium, boron, iron (III), titanium, zirconium and hafnium; (ii) R is C₁-C₆-alkyl, X is halogen or C₁₋₄-alkoxy; (iii) n is 0, 1 or 2, and m is 1, 2 or 3; an organic halide salt; and an organic base selected from the group consisting of: PPh₃, P(ortho-methylC₆H₄)₃, P(para-methylC₆H₄)₃, ClPPh₂, NPh₃, HNPh₂, P(OMe)₃, P(OPh)₃, Ph₂POPh, AsPh₃, SbPh₃, and BiRₓR′_{y} where x+y is 3 and R, R′ is alkyl, aryl, H, alkenyl, and alkynyl.

For example, M can be aluminum, gallium, boron or iron (III), or M titanium, zirconium, hafnium or aluminum. In particular, the buffered ionic liquid of claim 2 wherein M is aluminum, and the compound of the formula RₙMX₃, or of the formula RₘM₂X₆₋ₘ, is selected from the group consisting of aluminum halide, alkylaluminum dihalide, dialkylalumnum halide, trialkylaluminum, dialuminum trialkyl trihalide; dialkylaluminum alkoxide XAl(OR)₂, X₂Al(OR), Al(OR)₃, RAl(OR)₂, R₂Al(OR); and dialuminum hexahalide (AlX₆).

In some embodiments, the compound of the formula RₙMX₃, or of the formula RₘM₂X₆₋ₘ is selected from the group consisting of ethyl aluminum dichloride, dialuminum triethyl trichloride, diethyl aluminum ethoxide [(C₂H₅)₂Al(OC₂H₅)], trichloroaluminum (AlCl₃), trichloroaluminum dimer (Al₂Cl₆), diethyl aluminum chloride (Et₂AlCl), and triethyl aluminum (Et₃Al).

The organic halide salt can be a hydrocarbyl-substituted ammonium halide represented by the formula R⁴NR¹R²R³—Halide, wherein each of R¹, R², R³ and R⁴ is H or C₁-C₁₂ alkyl, hydrocarbyl substituted imidazolium halide; hydrocarbyl-substituted N-containing heterocycles selected from the group consisting of pyridinium, pyrrolidine, piperidine, and the like. For example, the organic halide salt can be selected from the group consisting of 1-alkyl-3-alkyl-imidazolium halides, alkyl pyridinium halides and alkylene pyridinium dihalides. The organic halide salt can also be selected from the group consisting of 1-methyl-3-ethyl imidazolium chloride, 1-ethyl-3-butyl imidazolium chloride, 1-methyl-3-butyl imidazolium chloride, 1methyl-3-butyl imidazolium bromide, 1-methyl-3-propyl imidazolium chloride, ethyl pyridinium chloride, ethyl pyridinium bromide, ethylene pyridinium dibromide, ethylene pyridinium dichloride, 4-methylpyridinium chloride, butyl pyridinium chloride and benzyl pyridinium bromide.

In some embodiments, the organic base is triphenylphosphine, triphenybismuthine or triphenylamine. The buffered ionic liquid can comprise BMIMCl (butylmethyl imidazolium chloride)/AlCl₃:PPh₃ in, for example, a ratio of about 0.05-1.5/1-2/0-0.5 by weight. The buffered ionic liquid can also comprise BMIMCl (butylmethyl imidazolium chloride)/AlCl₃/BiPh₃ in, for example, a ratio of about 0.05-1.5/1-2/0-0.5 by weight.

There is also provided herein an olefin dimerization process, comprising:

dimerizing olefins in the presence of a nickel catalyst in an buffered ionic liquid, comprising a compound of the formula RₙMX₃, or of the formula RₙM₂X₆₋ₘ, wherein:
i) M is a metal selected from the group consisting of aluminum, gallium, boron, iron (III), titanium, zirconium and hafnium;
ii) R is C₁-C₆-alkyl,
iii) X is halogen or C₁₋₄-alkoxy;
iv) n is 0, 1 or 2, and m is 1, 2 or 3;

an organic halide salt; and

an organic base selected from the group consisting of: PPh₃, P(ortho-methylC₆H₄)₃, P(para-methylC₆H₄)₃, ClPPh₂, NPh₃, HNPh₂, P(OMe)₃, P(OPh)₃, Ph₂POPh, AsPh₃, SbPh₃, and BiRₓR′_{y} where x+y is 3 and R, R′ is alkyl, aryl, H, alkenyl, and alkynyl;

and wherein said process results in at least 85% dimers. For example, the base can be triphenylphospine or triphenylbismuthine, the nickel catalyst can be

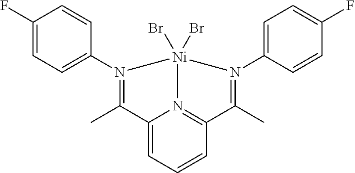

and, for example, about 8 equivalents of ethylaluminum dichloride is added per equivalent of catalyst.

Dimerizing can be carried out under anaerobic conditions. The buffered ionic liquid can further comprise a dehydrated silica material on which said buffered ionic liquid is supported. The silica material can be treated with ethylaluminum dichloride. The buffered ionic liquid can further comprise silica, alumina, titania, zirconia, mixed oxides or mixtures thereof on which said buffered ionic liquid is supported. The buffered ionic liquid can be loaded at 80 wt % of said silica support material weight, such as at 200 wt % of said silica support material weight. The dimerization process can further comprise adding at least 0.09 equivalents, for example 0.12 equivalents, triphenylbismuthine or diphenyl-Y-bismuthine, wherein Y is a polar or ionic substituent, following the dimerizing step.

This application further provides an olefin dimerization process comprising:

reacting one or more olefins in the presence of a nickel catalyst and a buffered ionic liquid consisting essentially of:

(a) an organic halide salt;

(b) an organic base selected from the group consisting of PPh₃, P(p-XC₆H₄)₃; P(m-XC₆H₄)₃, diphenylphosphinoferrocene, and triphenylphosphino-p-trimethylammonium iodide; and

(c) AlCl₃.

“Halogen” or “halo” refers to an element in Group VII of the periodic table, such as fluorine (F), chlorine (Cl), bromine (Br), or iodine (I). Halogens with a single negative charge have the suffix “-ide”: fluoride (F—), chloride (Cl—), bromide (Br—) and iodide (I.).

“Hydrocarbyl” refers to an organic substituent consisting of carbon and hydrogen atoms. The hydrocarbyl substituent can be substituted or unsubstituted, and/or branched or unbranched, and/or saturated or unsaturated. Hydrocarbyl groups include alkyl, alkenyl, and alkynyl groups. Generically, hydrocarbyl groups are often referred by the symbol “R”.

“Alkyl” refers to an organic substituent consisting of carbon and hydrogen atoms that are singly bonded to each other. The alkyl group can comprise, for example, 1 to 12 carbon atoms and be substituted or unsubstituted, and/or branched or unbranched. Examples of alkyl include, but are not limited to C₁₋₄-alkyl, such as methyl, ethyl, propyl, isopropyl, butyl, isobutyl, sec-butyl, and tert-butyl; or larger alkyl groups such as pentyl, neopentyl, hexyl, heptyl, octyl, nonyl, decyl, undecyl, and dodecyl. In some embodiments, the alkyl is a C₁₋₆-alkyl, for example a C₁₋₄-alkyl, a C₁₋₅-alkyl, C₂₋₆-alkyl or C₃₋₆-alkyl.

“Alkenyl” refers to an organic substituent consisting of carbon and hydrogen atoms that are singly bonded to each other and contain at least one carbon-carbon double bond (C═C). The alkenyl group can comprise, for example, 1 to 12 carbon atoms and be substituted or unsubstituted, and/or branched or unbranched. Examples of alkyl include, but are not limited to C₂₋₄-alkenyl, such as ethenyl, propenyl, and butenyl; or larger alkyl groups such as pentenyl, hexenyl, heptenyl, octenyl, nonenyl, decenyl, undecenyl, and dodecenyl.

“Alkynyl” refers to an organic substituent consisting of carbon and hydrogen atoms that are singly bonded to each other and contain at least one carbon-carbon triple bond. The alkynyl group can comprise, for example, 1 to 12 carbon atoms and be substituted or unsubstituted, and/or branched or unbranched. Examples of alkyl include, but are not limited to C₂₋₄-alkynyl, such as ethynyl (acetylenyl), propynyl (propragyl), and butynyl; or larger alkyl groups such as pentynyl, hexynyl, heptynyl, octynyl, nonynyl, decynyl, undecynyl, and dodecynyl.

“Alkylene” refers to a divalent fragment consisting of repeating methylene (—CH₂—) units. Examples of alkylenes include, but are not limited to, methylene (—CH₂—), ethylene (—CH₂CH₂—), propylene (—CH₂CH₂CH₂—), butylene (—CH₂CH₂CH₂CH₂—), hexylene, nonylene, and dodecylene. Alkylenes can be C₁-C₁₅-alkylenes, such as C₁-C₁₂-alkylene, C₃-alkylene, C₆-alkylene, C₉-alkylene, and C₁₂-alklyene.

“Alkoxy” refers to a substituent consisting of —O-alkyl. For example, a C₁₋₄-alkoxyl includes, but is not limited to, methoxy, ethoxy, propoxy, isopropoxy, butoxy, isobutoxy, sec-butoxy, and tert-butoxy. Other alkoxy groups include, but are not limited to, pentoxy, hexoxy, heptoxy, octoxy, nonoxy, decoxy, undecoxy, and dodecoxy.

“N-containing heterocycle” refers to a cyclic compound comprising carbon and at least one nitrogen atom in the ring. N-containing heterocycles can be aromatic or non-aromatic, and/or charged or neutral, and/or substituted or unsubstituted. Heterocycles can have for example, 3-, 4-, 5-, 6-, or 7-membered rings. Examples of aromatic N-containing heterocycles include, but are not limited to azirine, diazirine, azete, pyrrole, imidazole, imidazoline, pyrazole, pyrazoline, pyridine, diazine, triazine, tetrazine, azepine, diazepine, azocine. Examples of non-aromatic (aliphatic) N-containing heterocycles include, but are not limited to aziridine, azetidine, diazetidine, azolidine, imidazolidine, pyrazolidine, piperazine, azepane, and azocane. Examples of positively charged N-containing heterocycles include, but are not limit to, pyrrolium, imidazolium, imidazolinium, pyrazolium, pyzolinium, pyridinium, imidazolidinium, pyrazolidinium, and piperazinium.

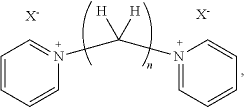

An alkylene pyridinium halide has the general formula wherein n is an integer, and each X″ is independently selected from F⁻, Cl⁻, Br⁻ and I⁻. Each X⁻ can be the same or different. The alkylene can be, for example, be a C₁-C₁₅-alkylene, such as C₁-C₁₂-alkylene, C₃-alkylene, C₆-alkylene, C₉-alkylene, and C₁₂-alklyene.

### Example 1

### Acidic Dimerization Reactions

In general, pressure Schlenk tubes (300 ml) were used for the reactions described throughout. The active liquid given into the Schlenk tube and 40-60 ml propene was condensed into the Schlenk tube (using liquid nitrogen). Stirring rate was about 1200/min. After the requisite reaction time the pressure was released and the weight difference determined. Temperature was controlled by a water bath. All C₆ fractions of the following propene dimerization reactions with catalyst A consisted of (±2%) 25% n-hexenes, 69% methylpentenes and 6% dimethylbutenes.

Original experiments established that ionic liquid dimerization of olefins could be improved with buffering with aryl and phenyl compounds of Bi, P, N, As, and Sb. In particular, it was discovered that acidic ionic liquids can be buffered with phosphines (e.g. triphenylphosphine) in comparable molar ratios to the nitrogen bases as described in WO9847616.

A systems with the composition of 1.00:1.20 (AlCl₃:BMIMCl) in combination with a PPh₃ buffer as buffer.

TABLE 1
Nickel-catalyzed dimerization reactions of propene in BiPh₃ buffered
chloroaluminate melts with different compositions (Reaction conditions:
catalyst A; [cat] = 10⁻⁵ mol/g_{liquid}; T = 25° C.; stirring rate = 1200 min⁻¹;
t = 60 min; products removed in vacuum after each run).

| | Composition | | | | | Volatile |
|---|---|---|---|---|---|---|
| Sys- | [BMIMCl]/ | Ionic | | | Dimers/ | Products |
| tem | [AlCl₃]/ | Liquid | Run | Product | Trimers | C₆ + |
| No. | [BiPh₃] | [g] | No. | [g] | [%] | C₉ [wt %] |
| 1 | 1.00/1.20/0.05 | 2.59 | 1 | >40.91 | Oil | |
| 2 | 1.00/1.20/0.09 | 2.84 | 1 | >33.56 | 85.7/5.8 | 87 |
| 3 | 1.00/1.20/0.12 | 3.90 | 1 | >31.08 | 93.1/6.4 | 98 |
| | | | 2 | >19.49 | | 95 |
| | | | 3 | >22.97 | | 73 |
| | | | 4 | >24.65 | | 63 |
| 4 | 1.00/1.20/0.30 | 3.47 | 1 | 23.26 | 96.0/3.4 | 98 |
| | | | 2 | Inactive | | |
| 5 | 1.00/1.30/0.12 | 2.99 | 1 | >23.86 | 81.8/11.4 | 88 |
| | | | 2 | >22.11 | | 68 |
| | | | 3 | >24.04 | | 30 |
| 6 | 1.00/1.50/0.12 | 2.62 | 1 | >27.69 | 74.1/13.4 | 83 |
| | | | 2 | 24.31 | | 22 |
| 7 | 1.00/1.50/0.18 | 2.73 | 1 | >32.63 | 83.2/12.7 | 95 |
| | | | 2 | 33.02 | | 71 |
| | | | 3 | 15.38 | | 50 |
| 8 | 1.00/2.00/0.12 | 2.77 | 1 | >31.57 | 74.8/16.6 | 87 |
| | | | 2 | >27.62 | | 73 |
| | | | 3 | >24.38 | | 71 |
| | | | 4 | 19.14 | | 63 |
| | | | 5 | 7.14 | | 51 |
| 9 | 1.00/2.00/0.18 | 2.89 | 1 | >35.50 | 79.5/15.2 | 91 |
| | | | 2 | >28.87 | | 89 |
| | | | 3 | >33.33 | | 76 |
| | | | 4 | 17.05 | | 56 |
| 10 | 1.00/2.00/0.24 | 2.99 | 1 | >22.14 | 83.3/12.9 | 96 |
| | | | 2 | >33.21 | | 86 |
| | | | 3 | >31.91 | | 66 |
| | | | 4 | >18.40 | | 76 |
| 11 | 1.00/2.00/0.30 | 3.29 | 1 | >30.71 | 85.5/11.1 | 95 |
| | | | 2 | >35.78 | | 92 |
| | | | 3 | >34.97 | | 89 |
| | | | 4 | >27.68 | | 76 |
| | | | 5 | 16.14 | | 44 |
| 12 | 1.00/2.00/0.60 | 2.62 | 1 | >30.07 | 90.6/8.5 | 98 |
| | | | 2 | >23.94 | | 96 |
| | | | 3 | >25.53 | | 95 |
| | | | 4 | >31.98 | | 93 |
| | | | 5 | >28.78 | | 77 |
| | | | 6 | >28.35 | | 80 |
| | | | 7 | 21.81 | | 81 |
| | | | 8 | 13.99 | | 82 |
| | | | 9 | 7.14 | | 95 |
| | | | 10 | 7.03 | | 93 |
| | | | 11 | 4.77 | | 93 |
| | | | 12 | 4.17 | | 92 |

Surprisingly, even the most acidic system could be buffered with an aluminum-imidazolium ratio of two with only 0.12 equivalents of BiPh₃ (see System No. 8). Also when the systems were more acidic, the experiments showed significantly longer lifetimes compared to the less acidic systems. System No. 12 was used for twelve experiments in a row. The first six experiments converted 100% of the propene present in the Schlenk tube. Then the activity dropped slightly after each run. The yield of dimers and trimers was very high in all experiments. A skilled artisan would anticipate that the systems would show improved performance (than that shown) when cleaner propene than 2.3 is used.

### Example 2

### Comparison with Non-Inventive Systems

In order to elaborate the advantages of the BiPh₃ buffer over DIFASOL™ a series of experiments with DIFASOL™ conditions and mixed systems was performed (Table 2). The highly active standard DIFASOL™ system (System No. 13), similar to that commercially used by the IFP with a different catalyst, produced 79.6% dimers and 17.3% trimers with catalyst A. If a more acidic DIFASOL™ composition was used (System No. 14) the EtAlCl₂ was not able to buffer the system properly yielding only 68.8% dimers. The even more acidic System No. 15 only produced 54.4% dimers. Also huge amounts of alkylaluminum compounds were leached into the product phase since the 2:1 system was already saturated with aluminum compounds. Leaching is a major problem of DIFASOL systems with higher alkylaluminum contents.

The addition of 0.12 equivalents of BiPh₃ to the standard DIFASOL System (resulting in System No. 16) reduced the activity greatly, but 96.8% dimers were produced, which is a tremendous improvement. By adding only 0.06 equivalents of BiPh₃ (System No. 17), the system was more active than with 0.12 equivalents, but still produced 94.6% dimers. With only 0.03 equivalents (System No. 18), the system converted all propene present in the Schlenk tube still with a high selectivity of 94.1% to C₆ hydrocarbons.

These experiments demonstrated that by adding very small amounts of BiPh₃ to a DIFASOL™-like system, selectivity can be increased by 15%. In addition, DIFASOL™ produced reasonable amounts of oligomers higher than C₉, but the BiPh₃-buffered DIFASOL™ system did not produce oligomers higher than C₉ at all. With 0.01 equivalents the selectivity dropped again to that of the standard DIFASOL™ system.

If a less acidic DIFASOL™ system is used (e.g., System No. 20), the system yielded 84.1% dimers. The addition of 0.03 equivalents BiPh₃ to System No. 20 (resulting in System No. 21) again improved the dimer yield to 94.1%. The results for System No. 22 demonstrated that a system exclusively buffered by 0.12 eq. BiPh₃ produced 93.1% dimers, much more than any DIFASOL system has been able to produce.

TABLE 2
Nickel-catalyzed dimerization reactions of propene in typical
DIFASOL ™-like systems with additional BiPh₃ (Reaction
conditions: catalyst A; [cat] = 10⁻⁵ mol/g_{liquid};
T = 25° C.; stirring rate = 1200 min⁻¹; t = 45 min).

| System | Composition [BMIMCl]/ | Ionic | Product | Dimers/ |
|---|---|---|---|---|
| No. | [AlCl₃]/[EtAlCl₂]/[BiPh₃] | Liquid [g] | [g] | Trimers [%] |
| 13 | 1.00/1.20/0.20/0 | 2.42 | >32.77 | 79.6/17.3 |
| 14 | 1.00/1.50/0.50/0 | 1.60 | >29.70 | 68.6/21.9 |
| 15 | 1.00/2.00/0.40/0 | 2.53 | >23.42 | 54.4/26.2 |
| 16 | 1.00/1.20/0.20/0.12 | 2.66 | 3.99 | 96.8/2.9 |
| 17 | 1.00/1.20/0.20/0.06 | 2.19 | 11.71 | 94.6/5.3 |
| 18 | 1.00/1.20/0.20/0.03 | 1.75 | >25.42 | 94.1/5.8 |
| 19 | 1.00/1.20/0.20/0.01 | 2.76 | >26.85 | 79.4/16.9 |
| 20 | 1.00/1.00/0.20/0 | 1.98 | >25.53 | 84.1/14.5 |
| 21 | 1.00/1.00/0.20/0.03 | 2.68 | 20.03 | 94.1/5.8 |
| 22 | 1.00/1.20/0/0.12 | 3.90 | >31.09 | 93.1/6.4 |

Subsequently, mixed 2:1 systems containing ethylaluminum groups as well as BiPh₃ as buffer were investigated for their lifetimes (See Table 3). Again all propene dimers and trimers were removed in vacuum before subsequent runs and their weight percentage in relation to the whole product weight was determined. System No. 23 with small amounts of buffer shows a C₆ selectivity of only 76%. According to the removed products, the selectivity dropped quite fast in the second and third run. System No. 24 showed a slightly better performance keeping its selectivity to C₆ and C₉ between 80 and 90 wt % over five runs. To date, the best mixed system (for longevity) identified was the highly buffered System No. 26, which was still active in its 14^{th} run. High lifetime and selectivity reduced the activity of the system due to the high amount of buffer.

TABLE 3
Nickel-catalyzed dimerization reactions of propene in highly acidic chloroaluminate melts
buffered by EtAlCl₂ and BiPh₃ (Reaction conditions: [BMIM]⁺[Al₂Cl₇]⁻ ionic liquid; catalyst A;
[cat] = 10⁻⁵ mol/g_{liquid}; T = 25° C.; stirring rate = 1200 min⁻¹; t = 60 min; products
removed in vacuum after each run).

| | | | | | | | Volatile |
|---|---|---|---|---|---|---|---|
| System | [BiPh₃]/ | [EtAlCl₂]/ | Ionic | Run | | Dimers/ | Products C₆ + |
| No. | [BMIMCl] | [BMIMCl] | Liquid [g] | No. | Product [g] | Trimers [%] | C₉ [wt %] |
| 23 | 0.12 | 0.02 | 2.62 | 1 | >33.59 | 76.0/16.5 | 90 |
| | | | | 2 | >35.04 | | 74 |
| | | | | 3 | >26.41 | | 69 |
| 24 | 0.12 | 0.20 | 2.63 | 1 | >30.38 | 78.6/14.8 | 89 |
| | | | | 2 | >25.31 | | 90 |
| | | | | 3 | >25.81 | | 93 |
| | | | | 4 | >26.54 | | 84 |
| | | | | 5 | >34.91 | | 84 |
| | | | | 6 | >22.49 | | 61 |
| 25 | 0.20 | 0.20 | 3.07 | 1 | >23.17 | 86.0/11.2 | 94 |
| | | | | 2 | >24.63 | | 92 |
| | | | | 3 | >27.36 | | 93 |
| | | | | 4 | >26.78 | | 96 |
| | | | | 5 | >30.00 | | 89 |
| | | | | 6 | >26.95 | | 90 |
| | | | | 7 | >25.36 | | 74 |
| | | | | 8 | 17.94 | | 54 |
| 26 | 0.60 | 0.20 | 4.00 | 1 | 19.05 | 93.3/6.4 | 99 |
| | | | | 2 | 12.96 | | 97 |
| | | | | 3 | 7.74 | | 98 |
| | | | | 4 | 7.11 | | 98 |
| | | | | 5 | 8.90 | | 93 |
| | | | | 6 | 11.38 | | 96 |
| | | | | 7 | 25.81 | | 96 |
| | | | | 8 | >26.25 | | 74 |
| | | | | 9 | >26.23 | | 73 |
| | | | | 10 | >21.94 | | 72 |
| | | | | 11 | 23.73 | | 69 |
| | | | | 12 | 11.52 | | 76 |
| | | | | 13 | 7.47 | | 74 |
| | | | | 14 | 4.93 | | 74 |

### Example 3

### Leaching Effects

The most promising BiPh₃ and mixed EtAlCl₂/BiPh₃ systems were tested again. This time the product phase was decanted after each run.

By decanting the products leaching effects can be investigated qualitatively by comparing the results to the previous experiments.

First, the highly buffered BiPh₃ system (System No. 27) was investigated. Initially the system produced 90.5% dimers. The second and third runs showed a similar selectivity of 88.2% and 85.4%, respectively. Run 4 only produced 65.0% dimers. By adding additional BiPh₃ the selectivity could be increased to 82.0%. After one run it dropped to 65.6% again. Adding another 0.12 equivalents of BiPh₃ yielded a dimer selectivity of 73.4% in run 7. Surprisingly with decreasing activity the selectivity increased again without the addition of more buffer (runs 8 and 9).

The mixed System No. 28 was also tested for leaching effects. Similarly to System No. 27, after three decanted runs, the selectivity dropped significantly from initially 84.3% to 72.3%.

Finally, a Wasserscheid system was also tested (System Nos. 29 and 30). Instead of using a 1.20:1.00 system as described in the patent, we used a very acidic 2:1 system buffered by 0.60 equivalents of N-methylpyrrole, since more acidic systems were expected to be active for a longer time. This system displayed a high activity and selectivity in its first run similarly to the bismuth systems. Surprisingly the selectivity increased after each run reaching 94.4% C₆ in run 4. Activity decreased after run 3. Run 5 unexpectedly yielded an oil with low dimer content while run 4 produced 94.4% dimers. The same system was used again with the difference that another 0.60 equivalents of methylpyrrole were added after run 4 (System No. 30), resulting in this system becoming almost completely inactive after the addition of the buffer.

The triphenylbismuth system, as well as the prior art Wasserscheid system were both very active and selective for many experiments. By steadily supplying BiPh₃ buffer the selectivity could be maintained at a high level. Certainly the systems based only on BiPh₃ appeared to be very promising for commercial applications.

TABLE 4
Nickel-catalyzed dimerization reactions of propene in highly acidic
chloroaluminate melts buffered by EtAlCl₂, BiPh₃ and
N-methylpyrrole (Reaction conditions: [BMIM]⁺[Al₂Cl₇]⁻ ionic
liquid; catalyst A; [cat] = 10⁻⁵ mol/g_{liquid}; T = 25° C.; stirring
rate = 1200 min⁻¹; t = 60 min; products decanted after each run).

| | | Ionic | | | Dimers/ |
|---|---|---|---|---|---|
| System | Buffer(s) | Liquid | Run | Product | Trimers |
| No. | [Buffer]/[BMIMCl] | [g] | No. | [g] | [%] |
| 27 | 0.60 BiPh₃ | 3.79 | 1 | >34.46 | 90.5/8.8 |
| | | | 2 | >31.51 | 88.2/9.4 |
| | | | 3 | >37.93 | 85.4/11.7 |
| | | | 4 | >32.51 | 65.0/18.1 |
| | +0.12 BiPh₃ | | 5 | >32.34 | 82.0/12.2 |
| | | | 6 | >32.00 | 65.6/10.8 |
| | +0.12 BiPh₃ | | 7 | >28.24 | 73.4/8.9 |
| | | | 8 | 21.92 | 76.9/6.4 |
| | | | 9 | 10.21 | 84.3/4.1 |
| 28 | 0.20 BiPh₃, 0.20 EtAlCl₂ | 3.07 | 1 | >29.07 | 84.3/12.4 |
| | | | 2 | >34.74 | 82.2/11.4 |
| | | | 3 | >34.06 | 80.1/13.8 |
| | | | 4 | >36.20 | 72.3/16.4 |
| | | | 5 | >25.80 | 56.4/21.6 |
| | | | 6 | >36.74 | 67.5/11.8 |
| | | | 7 | 18.42 | 49.6/6.2 |
| | | | 8 | 28.20 | Oil |
| 29 | 0.60 N-Methylpyrrole | 2.67 | 1 | >34.21 | 85.8/13.3 |
| | | | 2 | >24.99 | 90.2/9.3 |
| | | | 3 | >30.52 | 92.1/7.6 |
| | | | 4 | 14.37 | 94.4/5.2 |
| | | | 5 | 8.60 | Oil |
| 30 | 0.60 N-Methylpyrrole | 4.11 | 1 | >39.37 | 86.0/13.2 |
| | | | 2 | >40.60 | 91.6/8.0 |
| | | | 3 | >34.00 | 92.7/7.0 |
| | | | 4 | >45.09 | 93.4/6.4 |
| | +0.60 N-Methylpyrrole | | 5 | 0.62 | n.a. |

### Sample 4

### Buffers

In the examples above it is possible to efficiently buffer even highly acidic ionic liquids with aluminium to imidazolium ratio of 2. Such highly acidic systems showed improved lifetimes compared to the less acidic 1.20:1.00 systems.

Buffer (0.30 equivalents) was chosen and the already identified buffers NPh₃ and PPh₃ (System Nos. 30 and 31) as well as several substituted phosphines (System Nos. 36-42) were tested again. Results indicated that the buffering ability of all phosphines mainly depended on their solubility in the ionic liquids. The non-ionic phosphines did not dissolve completely in those compositions since they are very nonpolar. The fluoro-, chloro- and bromo-substituted triphenylphosphines (System Nos. 32, 33 and 34, respectively) produced higher dimer yields. Without being bound by any particular theory, it is believed that such higher dimer yields arise because of such compounds' improved solubility over normal triphenylphosphine. When oxygen was present anywhere in the buffer, the system failed to dimerize (e.g., p-methoxyphenyl diphenylphosphine (System No. 36) diphenylphosphinobenzene-3-sulfonic acid sodium salt (System No. 40) and methyl diphenylphosphinite (System No. 37, which in fact appeared to react with the ionic liquid).

Diphenylphosphino ferrocene (System No. 39) also acted as a buffer beating the result of PPh₃ due to its higher solubility. The dependence on the solubility becomes even clearer with System No. 41. We synthesized this triphenylphosphine derivative bearing a para-trimethylammonium iodide function specifically for this application (System No. 42). It turned out to be the most efficient phosphine buffer, since only phosphine dissolved completely in the liquid. Surprisingly, the same compound with a tetrafluoroborate anion (System No. 43) hardly dissolved in the liquid and displayed no buffering ability. Also the (formerly) synthesized BMIMCl-substituted diphenylphosphine was tested (System No. 46) with no success. Finally, BiI₃, BiF₃ and thiophene were tested, but were unsuitable for buffering the system (See, System Nos. 43, 44, and 47, respectively).

TABLE 5
Nickel-catalyzed dimerization reactions of propene in highly acidic chloroaluminate melts
with different buffers (Reaction conditions: composition [buffer]/[BMIM]⁺[Al₂Cl₇]⁻ = 0.30; catalyst
A; [cat] = 10⁻⁵ mol/g_{liquid}; T = 25° C.; stirring rate = 1200 min⁻¹; t = 60 min; products removed in
vacuum after each run).

| | | | | | | Volatile |
|---|---|---|---|---|---|---|
| | | Ionic | | | Dimers/ | Products C₆ + |
| No. | Buffer | Liquid [g] | Run No. | Product [g] | Trimers [%] | C₉ [wt %] |
| 31 | NPh₃ | 2.62 | 1 | >22.59 | 68.0/12.2 | 75 |
| | | | 2 | 18.15 | Oil | |
| 32 | PPh₃ | 2.25 | 1 | >27.29 | 57.4/17.9 | 62 |
| | | | 2 | >25.56 | | 54 |
| | | | 3 | 13.70 | | 12 |
| 33 | P(p-FC₆H₄)₃ | 2.43 | 1 | >33.30 | 64.2/13.5 | 72 |
| | | | 2 | >27.48 | | 62 |
| 34 | P(p-ClC₆H₄)₃ | 1.55 | 1 | 4.54 | 53.5/5.8 | 27 |
| 35 | P(p-BrC₆H₄)₃ | 2.66 | 1 | 19.66 | 76.2/7.7 | 75 |
| | | | 2 | 11.73 | | 67 |
| 36 | P(m-ClC₆H₄)₃ | 2.99 | 1 | 18.87 | 78.6/7.3 | 81 |
| | | | 2 | 2.17 | Oil | |
| 37 | Ph₂P(p-MeOC₆H₄) | 2.81 | 1 | 22.03 | Oil | |
| 38 | Ph₂POMe | 3.35 | 1 | >30.27 | Oil | |
| 39 | P(C₆F₅)₃ | 2.50 | 1 | 22.67 | Oil | |
| 40 | Ph₂PFc | 2.06 | 1 | >26.40 | 69.3/13.7 | 74 |
| | (where Fc = ferrocene) | | | | | |
| | | | 2 | 22.74 | n.a. | 59 |
| 41 | Ph₂P(m-NaSO₃C₆H₄) | 2.58 | 1 | 0.93 | Oil | |
| 42 | Ph₂P(p-I⁻Me₃N⁺C₆H₄) | 2.13 | 1 | 13.07 | 84.1/9.0 | 87 |
| | | | 2 | 11.10 | | 77 |
| | | | 3 | Inactive | | |
| 43 | Ph₂P(p-BF₄⁻Me₃NC₆H₄) | 2.64 | 1 | 30.65 | Oil | |
| 44 | Bil₃ | 2.79 | 1 | 22.37 | Oil | |
| 45 | BiF₃ | 2.74 | 1 | >27.28 | Oil | |
| 46 | Ph₂P-BMIMCl | 6.82 | 1 | 25.35 | Oil | |
| 47 | Thiophene (is | 5.21 | 1 | 7.12 | Oil | |
| | polymerized) | | | | | |

### Example 5

### Air Sensitivity

Because AlCl₃ and AlCl₃-based ionic liquids are extremely sensitive towards hydrolysis but inert to air, the BiPh₃ system was also tested for air stability (Table 6). An active system was evacuated and filled with dry air (System No. 49), and left in static dry air over night. The following dimerization reaction of propene yielded 58.1% dimers. Compared to the same system kept under inert gas (System No. 48) the selectivity was lower Adding BiPh₃ after the first run increased the dimer and trimer selectivity drastically.

In principal, the BiPh₃ systems are stable in air prolonged contact to air seemed to slowly oxidize the BiPh₃ to O═BiPh₃. Bi(V) does not possess a free electron pair and thus is unable to act as a buffer. The air stability was a major advantage over most other dimerization systems, which use alkylaluminum compounds and rapidly react with oxygen. Thus, the systems of the invention were easier to handle, and the propene did not have to be purified from oxygen completely before the reactions.

In System No. 49, the Schlenk tube was evacuated, filled with dry air and left standing for 30 minutes twice and then for 12 hours before run no. 1.

TABLE 6
Air stability of BiPh₃-buffered acidic chloroaluminate ionic liquid
dimerization systems (Reaction conditions: composition [BiPh₃]/
[BMIM]⁺[Al₂Cl₇]⁻ = 0.18; catalyst A; [cat] = 10⁻⁵ mol/g_{liquid}; T = 25° C.;
stirring rate = 1200 min⁻¹; t = 60 min; products removed in vacuum
before run 2, 0.12 BiPh₃ added before run 2).

| | | | | | | Volatile |
|---|---|---|---|---|---|---|
| | | Ionic | | | Dimers/ | Products |
| System | Gas | Liquid | Run | Product | Trimers | C₆ + |
| No. | Atmosphere | [g] | No. | [g] | [%] | C₉ [wt %] |
| 48 | Argon | 2.89 | 1 | >35.50 | 79.5/15.2 | 91 |
| 49 | Air | 2.85 | 1 | >32.21 | 58.1/11.7 | 37 |
| | | | 2 | >25.82 | | 89 |

### Example 6

### Solid Supports

In this example, we supported the bismuth-buffered system (BMIMCl/AlCl₃/BiPh₃=1/2/0.6) on a heterogeneous support material (Table 7) because of the obvious advantages of using solid supports.

First, an active system simply was coated on dehydrated Davicat™ SI1102 silica with different loadings (System No. 50 and 51). With 200 wt % the system already was greasy, with 150 wt % a free-flowing powder was obtained. Both loadings displayed low activities and selectivities. The bad performance may result from the interaction of the aluminium chloride in the ionic liquid with the surface OH-groups. Aluminum chloride is very oxophilic and probably reacts with such groups. Therefore, the silica was treated with ethylaluminum dichloride. The ethyl groups react with the surface OH-groups leaving an AlCl₂-capped silica surface behind. The excess EtAlCl₂ was washed out and the silica was dried and used as support material (System Nos. 52-58). Silica bearing AlCl₂ groups on its surface is a strong Lewis acid similar to the unbuffered ionic liquid system.

The highest possible loading of such modified systems was 120 wt % (System No. 52), above which the system became greasy. This system was active and produced 82.9% dimers with modest activity. Next 1.5:1 and 2:1 systems were tested with 100 wt % loading (System Nos. 53 and 54, respectively). The less acidic System No. 53 displayed low activity; the dimer selectivity was high-94.2%. The system was active for 10 runs before the dimer selectivity dropped significantly, although the product phase was decanted. The more acidic System No. 54 also was active for 10 runs showing a higher activity compared to System No. 53. The addition of BiPh₃ after run 10 resulted in an increased dimer selectivity and activity. The same system with a lower buffer content of only 0.30 equivalents (System No. 55) yielded only 65.6% dimers. The next step was to reduce the loading to 80 wt % (System Nos. 57 and 58). While the 2:1 system with 0.60 equivalents of buffer (System No. 57) produced only oil the same system with 1 equivalent of buffer (System No. 58) produced 90.4% dimers with modest activity.

The results indicate that the surface AlCl₂ groups further increased the overall acidity of the supported system. When highly active and selective biphasic dimerization systems were used, same selectivities on silica supported catalyst was only reached when higher amounts of buffer were used. Increased acidity also improved the systems' lifespan and reduced leaching. When selectivity decreased, it could be restored by adding more BiPh₃ (System Nos. 54 and 58). Unfortunately, the overall activity was lower compared to unsupported systems.

TABLE 7
Nickel-catalyzed dimerization reactions of propene in BiPh₃ buffered chloroaluminate
ionic liquid supported on Davicat ™ SI1102 silica and surface modified Davicat ™ SI1102
silica (Reaction conditions: catalyst A; [cat] = 10⁻⁵ mol/g_{liquid}; T = 25° C.; no stirring;
t = 60 min; products decanted after each run).

| | | | Ionic | | | | |
|---|---|---|---|---|---|---|---|
| | | | Liquid | | | | |
| | | | Loading | | | | |
| | Composition | | on | | | | |
| System | [BMIMCl]/[AlCl₃]/ | Support | Support | Ionic | Run | Product | Dimers/ |
| No. | [BiPh₃] | Material | [wt %] | Liquid [g] | No. | [g] | Trimers [%] |
| 50 | 1.00/2.00/0.60 | SiO₂ | 200 | 3.70 | 1 | 14.02 | 72.1/9.6 |
| | | (dehydrated) | | | | | |
| | | | | | 2 | 4.53 | 64.1/10.5 |
| 51 | 1.00/2.00/0.60 | SiO₂ | 150 | 2.74 | 1 | 6.00 | Oil |
| | | (dehydrated) | | | | | |
| 52 | 1.00/2.00/0.60 | SiO₂—AlCl₂ | 120 | 3.76 | 1 | 16.19 | 82.9/8.9 |
| 53 | 1.00/1.50/0.60 | SiO₂—AlCl₂ | 100 | 3.82 | 1 | 6.85 | 94.2/5.0 |
| | | | | | 2 | 6.22 | 93.0/5.8 |
| | | | | | 3 | 7.61 | 92.2/6.3 |
| | | | | | 4 | 9.46 | 88.3/8.0 |
| | | | | | 5 | 9.93 | 89.9/7.1 |
| | | | | | 6 | 10.85 | 85.0/7.8 |
| | | | | | 7 | 12.32 | 80.4/8.0 |
| | | | | | 8 | 10.76 | 72.8/9.6 |
| | | | | | 9 | 9.01 | 65.6/9.0 |
| | | | | | 10 | 7.76 | 58.2/7.7 |
| | +0.12 BiPh₃ | | | | 11 | 5.48 | 75.9/5.6 |
| 54 | 1.00/2.00/0.60 | SiO₂—AlCl₂ | 100 | 3.66 | 1 | 19.66 | 89.5/9.2 |
| | | | | | 2 | 12.67 | 89.2/8.7 |
| | | | | | 3 | 11.67 | 85.5/9.8 |
| | | | | | 4 | 10.73 | 88.6/8.0 |
| | | | | | 5 | 9.75 | 81.7/10.0 |
| | | | | | 6 | 11.78 | 84.2/8.6 |
| | | | | | 7 | 13.37 | 80.6/9.3 |
| | | | | | 8 | 10.36 | 76.9/10.2 |
| | | | | | 9 | 12.46 | 69.5/12.3 |
| | | | | | 10 | 9.06 | 56.9/12.3 |
| | +0.12 BiPh₃ | | | | 11 | 13.89 | 84.0/6.1 |
| 55 | 1.00/2.00/0.45 | SiO₂—AlCl₂ | 100 | 3.00 | 1 | 20.88 | 67.5/14.4 |
| | | | | | 2 | 15.20 | 65.7/13.4 |
| | | | | | 3 | 8.89 | 62.4/10.9 |
| | | | | | 4 | 8.17 | 59.6/9.2 |
| 56 | 1.00/2.00/0.30 | SiO₂—AlCl₂ | 100 | 3.14 | 1 | 21.84 | 65.6/15.1 |
| 57 | 1.00/2.00/0.60 | SiO₂—AlCl₂ | 80 | 3.70 | 1 | 24.09 | Oil |
| 58 | 1.00/2.00/1.00 | SiO₂—AlCl₂ | 80 | 2.72 | 1 | 13.22 | 89.6/8.2 |
| | | | | | 2 | 9.51 | 89.4/8.1 |
| | | | | | 3 | 9.42 | 90.0/7.8 |
| | | | | | 4 | 8.56 | 89.7/7.8 |
| | | | | | 5 | 9.20 | 89.8/7.4 |
| | | | | | 6 | 10.01 | 85.6/8.4 |
| | | | | | 7 | 10.06 | 75.7/11.3 |
| | | | | | 8 | 9.46 | 74.4/11.9 |
| | | | | | 9 | 7.32 | 68.5/12.9 |
| | +0.30 BiPh₃ | | | | 10 | 2.28 | 81.1/8.9 |

The support was changed from silica to high density polyethylene (HDPE).

The systems started dimerizing with a loading (of ionic liquid on the support) of around 150 wt % (System No. 59), with 350 wt % loading the HDPE became greasy (System No. 68). The typical 1/2/0.60 system supported on HDPE was less selective compared to the unsupported system. With 150 wt % loading only 64.3% dimers were produced (System No. 59), and with 200 wt % loading 68.0% dimers were produced. With 300 wt % loading (System No. 67) 85.3% dimers were produced. This result suggests that at the interface between the ionic liquid and the support material there are interactions that reduce the ability of BiPh₃ to buffer the systems sufficiently.

When less acidic 1.5:1 systems were used (System Nos. 62-65) with different loadings, the dimer yields were higher. Repeatability of the systems was poor compared to the silica supported systems described above. The activity was also reduced drastically compared to the corresponding unsupported systems. High loadings were necessary and due to the nonpolarity of HDPE, the yellow product phase indicated that some of the liquid was washed off steadily. Therefore, HDPE and probably all nonpolar hydrocarbons are not suitable as support material.

TABLE 8
Nickel-catalyzed dimerization reactions of propene in BiPh₃ buffered
chloroaluminate ionic liquid supported on high density polyethylene
(HDPE) (Reaction conditions: catalyst A; [cat] = 10⁻⁵ mol/g_{liquid};
T = 25° C.; no stirring; t = 60 min; products decanted after each run).

| | | Ionic | | | | |
|---|---|---|---|---|---|---|
| | | Liquid | | | | |
| | Composition | Loading | | | | |
| | [BMIMCl]/ | on | Ionic | | | Dimers/ |
| System | [AlCl₃]/ | Support | Liquid | Run | Product | Trimers |
| No. | [BiPh₃] | [wt %] | [g] | No. | [g] | [%] |
| 59 | 1.00/2.00/0.60 | 150 | 3.70 | 1 | 32.18 | 64.3/9.0 |
| 60 | 1.00/2.00/0.30 | 200 | 2.92 | 1 | 22.59 | Oil |
| 61 | 1.00/2.00/0.60 | 200 | 3.70 | 1 | >31.52 | 68.0/14.4 |
| | | | | 2 | >30.04 | 67.3/11.1 |
| | | | | 3 | 15.94 | 71.4/7.9 |
| | | | | 4 | 4.07 | 66.1/9.7 |
| 62 | 1.00/1.50/0.12 | 200 | 2.62 | 1 | 5.97 | Oil |
| 63 | 1.00/1.50/0.60 | 200 | 3.91 | 1 | 17.10 | 94.5/3.8 |
| | | | | 2 | Inactive | |
| 64 | 1.00/1.50/0.30 | 300 | 3.00 | 1 | >22.83 | 76.4/7.5 |
| | | | | 2 | 13.45 | 70.7/6.1 |
| 65 | 1.00/1.50/0.60 | 300 | 3.86 | 1 | 10.90 | 96.0/3.0 |
| | | | | 2 | Inactive | |
| 66 | 1.00/2.00/0.30 | 300 | 2.99 | 1 | >31.28 | 53.7/7.9 |
| 67 | 1.00/2.00/0.60 | 300 | 3.81 | 1 | >33.69 | 85.3/10.2 |
| | | | | 2 | >26.92 | 61.8/16.7 |
| | | | | 3 | 31.87 | 65.5/12.2 |
| | | | | 4 | 16.87 | 68.7/8.5 |
| | | | | 5 | 5.90 | 67.2/6.0 |
| 68 | 1.00/2.00/0.60 | 350 | 3.70 | 1 | >33.19 | 68.8/13.8 |
| | | | | 2 | >29.12 | 69.6/12.1 |
| | | | | 3 | 13.09 | 68.6/7.5 |

### Example 7

### ZrCl₄ Effect

Use of ZrCl₄ instead of AlCl₃ in acidic ionic liquids was investigated. Neutral chloroaluminate liquids, to which 0.24 equivalents of ZrCl₄ were added, could be buffered by BiPh₃. This system was also active for the dimerization of propene (System No. 74). The more acidic System Nos. 75-77 were also tested. Those systems did not dimerize propene. When higher amounts of BiPh₃ were used the systems became inactive (System Nos. 75 and 77), with less buffer only viscous oils were produced (System No. 76). Therefore, the substitution of AlCl₃ by ZrCl₄ was possible to some extent but did not have advantages.

TABLE 9
Nickel-catalyzed dimerization reactions of propene in neutral
chloroaluminate melts containing ZrCl₄ and BiPh₃ as buffer (Reaction
conditions: catalyst A; [cat] = 10⁻⁵ mol/g_{liquid}; T = 25° C.; stirring
rate = 1200 min⁻¹; t = 60 min).

| | | Ionic | | Dimers/ |
|---|---|---|---|---|
| System | Composition | Liquid | Product | Trimers |
| No. | [BMIM]⁺[AlCl₄]⁻/[ZrCl₄]/[BiPh₃] | [g] | [g] | [%] |
| 74 | 1.00/0.24/0.14 | 2.52 | 11.92 | 93.0/5.0 |
| 75 | 1.00/0.80/0.30 | 3.99 | Inactive | n.a. |
| 76 | 1.00/0.80/0.12 | 3.82 | 17.87 | Oil |
| 77 | 1.00/0.40/0.12 | 3.52 | Inactive | n.a. |

### Example 8

### Cations

Table 10 illustrates the results of nickel catalyzed dimerization reactions of propene in chloroaluminate melts with different quaternary ammonium cations and BiPh₃ acting as buffer. The cations used in the runs illustrated in Table 10 are shown in FIG. 3 with the Cation No. corresponding to the Cation No. in Table 10.

The data illustrated in Table 10 shows that principally all liquids based on the quaternary ammonium salts (1-13) can be used in dimerization systems yielding between 80 and 90% dimers in the first catalytic experiment. Only cation 6 decomposed during the reaction. Most of the cations improved upon the performance of the standard system 12.

The main differences between the illustrated systems can be observed in the repetitions of the catalytic experiment. The best combination of selectivity and repeatability shows the trimethylanilinium cation 7 followed by benzyltributylammonium 3 and benzylcyclohexyldimethylammonium 4. The differences result probably due to a better solubility of BiPh₃ in those liquids, reducing leaching effects.

TABLE 10
Nickel-catalyzed dimerization reactions of propene in BiPh₃ buffered chloroaluminate
melts based on quaternary ammonium cations (Reaction conditions: composition [BiPh₃]/
[cation]⁺[Al₂Cl₇]⁻ = 0.30; catalyst A; [cat] = 10⁻⁵ mol/g_{liquid}; T = 25° C.; stirring rate = 1200 min⁻¹;
t = 60 min; products decanted after each run).

| | Ionic | Run 1 | | | | | | | |
|---|---|---|---|---|---|---|---|---|---|
| Cation | Liquid | C₆ [%] | | | | | | | |
| No. | [g] | Product [g] | Run 2 | Run 3 | Run 4 | Run 5 | Run 6 | Run 7 | Run 8 |
| 1 | 2.74 | 89.2 | 86.6 | 84.9 | 75.4 | 64.5 | | | |
| | | >31.62 | >34.40 | >35.15 | >35.39 | 24.08 | | | |
| 2 | 2.65 | 88.9 | 89.1 | 78.2 | 63.5 | | | | |
| | | >28.14 | >28.77 | >31.66 | >26.72 | | | | |
| 3 | 2.77 | 90.3 | 90.0 | 86.0 | 73.6 | 72.3 | 69.3 | 65.2 | |
| | | >26.34 | >31.39 | >37.61 | >36.31 | 24.49 | 17.74 | 15.03 | |
| 4 | 3.66 | 89.6 | 90.1 | 86.0 | 79.7 | 65.1 | 72.8 | 55.5 | |
| | | >28.64 | >32.63 | >36.85 | >33.67 | >27.77 | >28.19 | 18.51 | |
| 5 | 2.73 | 89.0 | 84.1 | 77.3 | 59.1 | | | | |
| | | 22.67 | >28.99 | 22.05 | 23.98 | | | | |
| 6 | 3.93 | Oil (decomp.) | | | | | | | |
| | | 28.54 | | | | | | | |
| 7 | 2.96 | 89.7 | 89.9 | 88.8 | 85.9 | 77.8 | 75.4 | 74.0 | 66.9 |
| | | >38.39 | >42.25 | >42.69 | >37.73 | >42.94 | >41.46 | 22.64 | 13.59 |
| 8 | 2.63 | 89.3 | 86.4 | 85.1 | 81.8 | 58.0 | 55.9 | | |
| | | >30.21 | >31.42 | >33.13 | >35.14 | 22.34 | 21.13 | | |
| 9 | 3.46 | 88.6 | 84.3 | 81.0 | 64.0 | | | | |
| | | >32.28 | >31.11 | >38.74 | >30.97 | | | | |
| 10 | 3.25 | 80.3 | 55.9 | | | | | | |
| | | >32.46 | 15.27 | | | | | | |
| 11 | 3.04 | 95.1 | 93.5 | 87.6 | 70.2 | 69.3 | 62.5 | | |
| | | 18.27 | 22.17 | >30.71 | 28.77 | 27.05 | 22.75 | | |
| 12 | 2.78 | 86.2 | 85.8 | 81.4 | 68.7 | 63.5 | | | |
| | | >28.23 | >31.18 | >34.05 | >34.95 | 24.97 | | | |
| 13 | 2.89 | 87.3 | 69.3 | 73.8 | 66.5 | | | | |
| | | 23.17 | 21.67 | 20.81 | 28.16 | | | | |

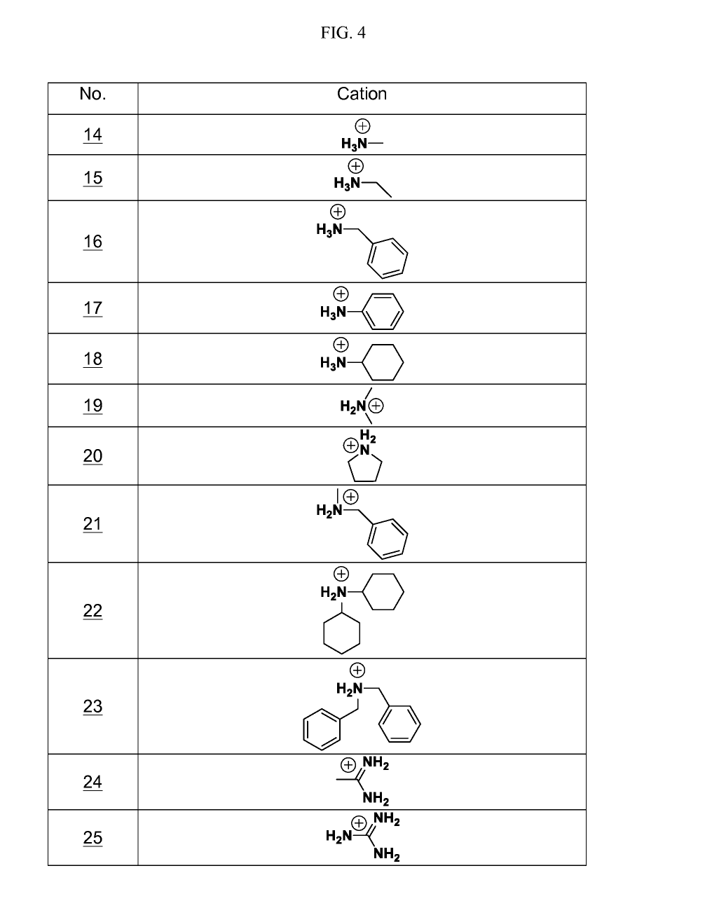

Table 11 shows the results of the propene dimerization reactions with hydrochloride salts of primary and secondary amines as cations. More specifically, Table 11 illustrates the results of nickel catalyzed dimerization reactions of propene in chloroaluminate melts with different hydrochloride salts of primary/secondary amines and BiPh₃ acting as buffer [(Composition [Cation]⁺[Al₂Cl₇]⁻/BiPh₃=1.00/0.30, catalyst concentration 0.01 mmol_{catalyst}/ml_{liquid} at 25° C., catalyst A, reaction time 60 minutes, products decanted after each run, constant stirring rate)]. The cations used in the runs illustrated in Table 11 are shown in FIG. 4 with the Cation no. corresponding to the Cation No. in Table 11.

Most unbuffered ionic liquids were solids at room temperature, except pyrrolidine hydrochloride (7) and acetamidine hydrochloride (11) based liquid. Cations 14, 15, 17, 19, 24 and 25 were purchased; all others were synthesized by adding concentrated aqueous hydrochloric acid to the corresponding amines following by vacuum drying at elevated temperatures.

Surprisingly, the hydrochloride salts can be used for dimerization systems. Systems with alkylaluminum compounds like DIFASOL™ must not contain acidic protons, because those would instantly react with the alkyl groups. Also Wasserscheid only used standard quaternary 1-butyl-3-methylimidazolium salts.

Systems based on hydrochloride salts of simple primary or secondary amines do not show the high selectivity achieved with quaternary ammonium cations, as illustrated in Table 11.

TABLE 11
Nickel-catalyzed dimerization reactions of propene in BiPh₃ buffered chloroaluminate
melts based on hydrochloride salts of primary and secondary amines (Reaction conditions:
composition [BiPh₃]/[cation]⁺[Al₂Cl₇]⁻ = 0.30; catalyst A; [cat] = 10⁻⁵ mol/g_{liquid}; T = 25° C.; stirring
rate = 1200 min⁻¹; t = 60 min; products decanted after each run).

| | | Run 1 | | | | | | | | |
|---|---|---|---|---|---|---|---|---|---|---|
| | Ionic | C₆ [%] | | | | | | | | |
| Cation | Liquid | Product | | | | | | | | |
| No. | [g] | [g] | Run 2 | Run 3 | Run 4 | Run 5 | Run 6 | Run 7 | Run 8 | Run 9 |
| 14 | 2.74 | 59.7 | 68.0 | 67.2 | 72.6 | 73.5 | 73.4 | 62.1 | | |
| | | >35.06 | >5.88 | >3.08 | >36.96 | 30.32 | 16.04 | 13.00 | | |
| 15 | 3.83 | 79.0 | 79.3 | 74.1 | 65.2 | 67.4 | 71.5 | 67.5 | 67.3 | |
| | | 22.88 | 22.66 | >6.34 | >7.01 | 26.07 | 25.02 | 20.68 | 12.69 | |
| 16 | 3.58 | 66.1 | 68.1 | 72.1 | 69.5 | 69.3 | 49.7 | Oil | | |
| | | >35.01 | >5.62 | 27.29 | 22.70 | 16.57 | 16.18 | 13.84 | | |
| 17 | 2.82 | Oil | | | | | | | | |
| | | 19.12 | | | | | | | | |
| 18 | 3.21 | 76.1 | 72.6 | 70.2 | 70.1 | 62.0 | | | | |
| | | >28.36 | >34.60 | >32.10 | >29.95 | 27.68 | | | | |
| 19 | 4.00 | 73.3 | 71.5 | 76.6 | 69.5 | 68.1 | 60.7 | 65.6 | 66.5 | 69.2 |
| | | >31.55 | >31.11 | >31.76 | >30.71 | >32.48 | >33.44 | >32.47 | >28.24 | 23.37 |
| 20 | 3.08 | 83.0 | 84.1 | 78.5 | 70.0 | 69.9 | 65.0 | | | |
| | | 9.59 | 12.63 | 15.89 | 26.21 | 19.94 | 15.33 | | | |
| 21 | 2.96 | 64.3 | 63.6 | Oil | | | | | | |
| | | >40.82 | >35.83 | >29.26 | | | | | | |
| 22 | 3.01 | 90.4 | 88.5 | 88.5 | 70.4 | 66.5 | 63.4 | | | |
| | | >27.43 | >24.54 | 29.66 | >34.75 | >29.88 | 20.20 | | | |
| 23 | 3.13 | 73.0 | 65.0 | 61.3 | | | | | | |
| | | >29.95 | >33.11 | >28.83 | | | | | | |
| 24 | 3.71 | 67.7 | 62.3 | 60.4 | 57.0 | Oil | | | | |
| | | >37.38 | >31.03 | 30.78 | 21.81 | 20.05 | | | | |
| 25 | 2.68 | Oil | | | | | | | | |
| | | 17.51 | | | | | | | | |

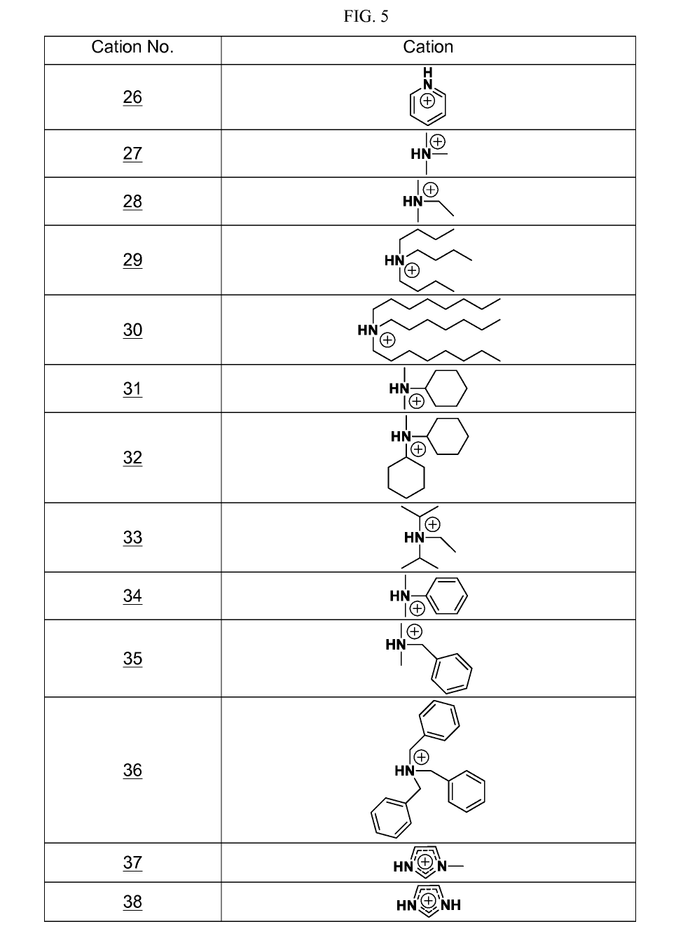

Because hydrochloride salts of primary and secondary amines in principle can be used in BiPh₃ buffered dimerization systems now hydrochloride salts of tertiary amines were also screened. Table 12 illustrates the results of nickel-catalyzed dimerization reactions of propene in chloroaluminate melts with different hydrochloride salts of tertiary amines and BiPh₃ acting as buffer (Composition [Cation]⁺[Al₂Cl₇]⁻/BiPh₃=1.00/0.30, catalyst concentration 0.01 mmol_{catalyst}/ml_{liquid} at 25° C., catalyst A, reaction time 60 minutes, products decanted after each run, constant stirring rate). The cations used in the runs illustrated in Table 12 are shown in FIG. 5 with the Cation no. corresponding to the Cation No. in Table 12. Cations 26, 27 and 37 purchased, 34 was synthesized with HCl gas from N,N-dimethylaniline. The rest was obtained from the free amines and concentrated aqueous HCl. While many cations produced around 90% propene dimers in the first experiment, amines with sterically demanding, long chain substituents were superior in terms or repeatability. Especially tributylamine hydrochloride (29), trioctylamine hydrochloride (30), dimethylcyclohexyl amine hydrochloride (31), dicyclohexylmethylamine hydrochloride (32) and the hydrochloride salt of the sterically demanding Hunig's base (33) displayed an excellent performance. 32 maintained an excellent selectivity as well as a high activity over 8 catalytic runs, after the addition of small amounts of buffer the selectivity could be increased again in runs 9-14.

TABLE 12
Nickel-catalyzed dimerization reactions of propene in BiPh₃ buffered chloroaluminate
melts based on hydrochloride salts of tertiary amines (Reaction conditions: composition [BiPh₃]/
[cation]⁺[Al₂Cl₇]⁻ = 0.30; catalyst A; [cat] = 10⁻⁵ mol/g_{liquid}; T = 25° C.; stirring
rate = 1200 min⁻¹; t = 60 min; products decanted after each run).

| | | Run 1 | | | | | | | | |
|---|---|---|---|---|---|---|---|---|---|---|
| | Ionic | C₆ [%] | | | | | | | | |
| | Liquid | Product | | | | | | | | |
| No. | [g] | [g] | Run 2 | Run 3 | Run 4 | Run 5 | Run 6 | Run 7 | Run 8 | Run 9 |
| 26 | 3.27 | 53.7 | | | | | | | | |
| | | 25.96 | | | | | | | | |
| 27 | 3.23 | 89.6 | 86.3 | 82.6 | 69.4 | Oil | | | | |
| | | 24.53 | >27.94 | >31.33 | >30.43 | >25.90 | | | | |
| 28 | 3.10 | 90.0 | 90.6 | 77.5 | 60.4 | | | | | |
| | | >27.35 | >26.64 | >34.96 | >25.63 | | | | | |
| 29 | 3.28 | 89.4 | 90.0 | 89.8 | 88.3 | 86.3 | 77.1 | 70.7 | 64.3 | |
| | | >34.65 | >36.32 | >35.76 | >34.81 | >41.79 | >37.70 | 28.23 | 13.57 | |
| 30 | 3.44 | 92.0 | 91.3 | 89.5 | 86.1 | 75.2 | 66.2 | | | |
| | | 27.13 | 26.58 | 29.47 | >31.26 | 24.56 | 15.44 | | | |
| 31 | 2.75 | 87.9 | 88.3 | 87.5 | 87.4 | 83.8 | 76.0 | 66.8 | | |
| | | >34.25 | >33.46 | >33.82 | >33.69 | >35.31 | >35.49 | 22.71 | | |
| 32 | 3.13 | 88.0 | 89.7 | 89.4 | 88.5 | 87.8 | 83.6 | 76.6 | 70.5 | +0.20 |
| | | >26.95 | >29.66 | >30.30 | >31.77 | >36.04 | >32.38 | >33.93 | >28.89 | BiPh₃ |
| | | Run 9 | Run 10 | Run 11 | Run 12 | Run 13 | | Run 14 | | |
| | | 92.3 | 95.3 | 91.3 | 83.0 | 77.7 | +0.20 | 88.0 | | |
| | | 18.74 | 15.83 | >26.49 | >29.27 | 26.87 | BiPh₃ | 11.02 | | |
| 33 | 3.29 | 90.7 | 90.0 | 90.4 | 90.0 | 87.7 | 85.7 | 72.2 | 64.4 | +0.15 |
| | | >33.12 | >32.89 | >33.48 | >33.11 | >34.52 | >38.88 | >33.87 | >24.41 | BiPh₃ |
| | | Run 9 | Run 10 | | | | | | | |
| | | 92.7 | 93.7 | | | | | | | |
| | | 11.01 | 9.76 | | | | | | | |
| 34 | 3.35 | 63.1 | 68.4 | 74.0 | 70.3 | 73.3 | 66.6 | | | |
| | | >32.09 | >27.63 | 21.06 | 21.74 | 19.05 | 15.47 | | | |
| 35 | 2.64 | 87.8 | 87.1 | 83.2 | Oil | | | | | |
| | | >25.24 | >27.85 | >28.09 | >11.82 | | | | | |
| 36 | 3.17 | 73.5 | 73.4 | 71.6 | 70.3 | 70.2 | 67.1 | | | |
| | | 24.23 | 27.21 | >28.35 | >33.17 | >31.79 | 16.23 | | | |
| 37 | 3.50 | 84.0 | 74.4 | 64.4 | | | | | | |
| | | >37.93 | >38.31 | >29.44 | | | | | | |
| 38 | 3.58 | 75.5 | 67.5 | 70.7 | 69.7 | 69.6 | 73.4 | 71.4 | 68.0 | |
| | | >41.95 | >36.43 | >38.62 | >33.78 | 26.73 | 24.70 | 20.27 | 13.03 | |

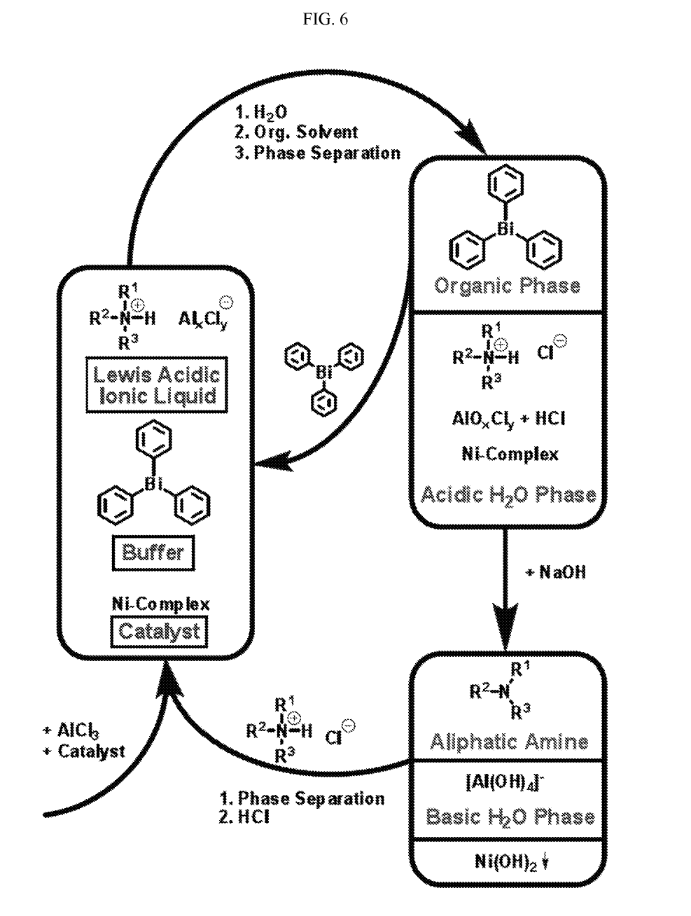

The use of hydrochloride salts has not only the advantage that they are very inexpensive, it also facilitates recycling depleted ionic liquid systems. That is, the amines can be recovered by a simple pH change. FIG. 6 illustrates a possible recycle scheme for a propene dimerizing ionic liquid system based on nonpolar aliphatic hydrochloride salts of tertiary amines. If an aliphatic amine with sufficiently long alkyl chains is used, the amine is insoluble in water and may be decanted in slightly basic media, for example, in those embodiments that tributylamine, trioctylamine, or methyldicyclohexylamine is used. Also, the water insoluble BiPh₃ can be extracted from the hydrolyzed liquid with any suitable organic solvent. Only very low cost AlCl₃ is consumed.

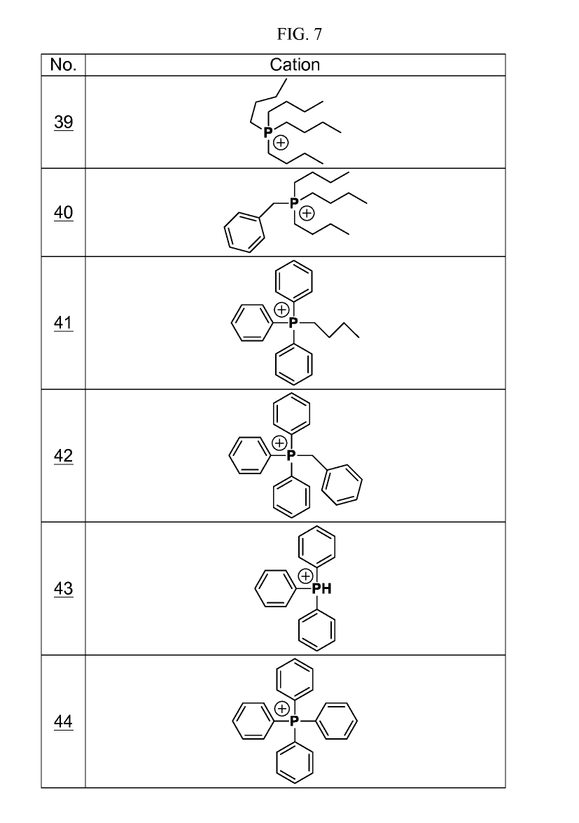

In addition to ammonium-based systems, phosphonium salts can also be used to form chloroaluminate ionic liquids. Thus, a series of phosphonium chloride salts was screened for their performance in the dimerization reaction of propene. Table 13 illustrates the results of nickel-catalyzed dimerization reactions of propene in chloroaluminate melts with different phosphonium chlorides and BiPh₃ acting as buffer (Composition [Cation]⁺[Al₂Cl₇]⁻/BiPh₃=1.00/0.30, catalyst concentration 0.01 mmol_{catalyst}/ml_{liquid} at 25° C., catalyst A, reaction time 60 minutes, products decanted after each run, constant stirring rate). The cations used in the runs illustrated in Table 13 are shown in FIG. 7 with the Cation no. corresponding to the Cation No. in Table 13.

Cations 39, 41 and 44 were purchased, 43 was obtained from triphenylphosphine and HCl gas in dry ether. The rest was obtained by benzylation with benzylchloride from the corresponding phosphines.

Benzyltributylphosphonium chloride (40) and triphenylbenzylphosphonium chloride (42) gave the best results in terms of selectivity, activity and repeatability. Due to the easy recycling of amine hydrochloride salts, those cations are preferred.

TABLE 13
Nickel-catalyzed dimerization reactions of propene in BiPh₃ buffered chloroaluminate
melts based on phosphonium cations (Reaction conditions: composition [BiPh₃]/
[cation]⁺[Al₂Cl₇]⁻ = 0.30; catalyst A; [cat] = 10⁻⁵ mol/g_{liquid}; T = 25° C.;
stirring rate = 1200 min⁻¹; t = 60 min; products decanted after each run).

| | Ionic | Run 1 | | | | | | |
|---|---|---|---|---|---|---|---|---|
| | Liquid | C₆ [%] | | | | | | |
| No. | [g] | Product [g] | Run 2 | Run 3 | Run 4 | Run 5 | Run 6 | Run 7 |
| 39 | 2.06 | 85.4 | 73.0 | 64.0 | 67.4 | Oil | | |
| | | >38.60 | >38.20 | 29.13 | 23.29 | 14.25 | | |
| 40 | 2.91 | 92.5 | 91.1 | 89.1 | 79.0 | 76.1 | 73.9 | Oil |
| | | >25.01 | >34.99 | >33.33 | >36.29 | >30.72 | 24.58 | 12.16 |
| 41 | 3.10 | 91.0 | 86.5 | 80.1 | 71.0 | Oil | | |
| | | >35.38 | >36.29 | 32.34 | 15.78 | 9.94 | | |
| 42 | 2.99 | 89.4 | 89.5 | 89.4 | 86.9 | 75.7 | 75.1 | 68.8 |
| | | >26.42 | >29.95 | >33.57 | >38.31 | >37.75 | 24.80 | 13.49 |
| 43 | 2.54 | 68.6 | 63.1 | 63.0 | 63.6 | 63.7 | 63.4 | |
| | | >28.08 | >30.36 | >29.91 | >38.89 | >27.24 | 14.90 | |
| 44 | 3.15 | 78.1 | 77.8 | 70.4 | 65.9 | | | |
| | | >31.81 | >36.78 | >31.96 | >24.95 | | | |

Improvement of the dimer selectivity of a DIFASOL™ system from 80% to 94% by the addition of small amounts of BiPh₃ is illustrated in Table 14. More particularly, Table 14 illustrates the results of nickel catalyzed dimerization reactions of propene in typical DIFASOL™-like systems with additional BiPh₃ and substituted triphenylphosphine B (catalyst concentration 0.01 mmol_{catalyst}/ml_{liquid} at 25° C., catalyst A, reaction time 45 minutes, constant stirring rate).

The effect of previously synthesized triphenylphosphine derivative B:

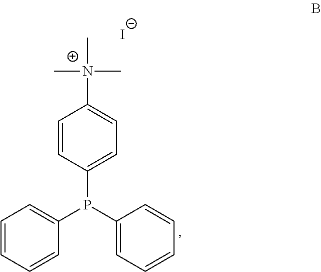

on the performance of such a DIFASOL™ system was investigated. By adding 0.03 equivalents 50 the C₆ selectivity could be increased slightly to 83.6%. 0.06 equivalents of B resulted in 89.1% dimers (50), an increase of about 10% compared to the standard system. The combination of DIFASOL™ and buffer yielded highly active and way more selective systems compared to a standard DIFASOL™ system. The results show the potential of triphenylphosphine, and the potential of triphenylbismuth to a lesser extent. Such an improvement is particularly evident when ionic substituents such as trimethylammonium groups are introduced, as such compounds remain in the liquid phase and cannot be leached into the product phase.

TABLE 14
Nickel-catalyzed dimerization reactions of propene in typical
DIFASOL ™-like systems with additional BiPh₃ and substituted
triphenylphosphine B (Reaction conditions: composition [BMIMCl]/
[AlCl₃]/[EtAlCl₂] = 1.00/1.20/0.20; catalyst A; [cat] =
10⁻⁵ mol/g_{liquid}; T = 25° C.; stirring rate = 1200 min⁻¹;
t = 45 min; products decanted after each run).

| | | | Ionic | Product | |
|---|---|---|---|---|---|
| No. | Buffer | [Buffer]/[BMIMCl] | Liquid [g] | [g] | Dimers [%] |
| 45 | — | — | 2.42 | >32.77 | 79.6 |
| 46 | BiPh₃ | 0.06 | 2.19 | 11.71 | 94.6 |
| 47 | BiPh₃ | 0.03 | 1.75 | >25.42 | 94.1 |
| 48 | BiPh₃ | 0.01 | 2.76 | >26.85 | 79.4 |
| 49 | B | 0.06 | 2.74 | >27.77 | 89.1 |
| 50 | B | 0.03 | 2.23 | >37.55 | 83.6 |

While a number of particular embodiments of the present invention have been described herein, it is understood that various changes, additions, modifications, and adaptations may be made without departing from the scope of the present invention, as set forth in the following claims.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The following references are incorporated by reference in their entirety:
WO9847616.

1. A buffered ionic liquid comprising:
a compound of the formula RₙMX₃₋ₙ or of the formula RₘM₂X₆₋ₘ, wherein:
i) M is a metal selected from the group consisting of aluminum, gallium, boron, iron (III), titanium, zirconium and hafnium;
ii) R is C₁-C₆-alkyl,
iii) X is halogen or C₁₋₄-alkoxy;
iv) n is 0, 1 or 2, and m is 1, 2 or 3;
an organic halide salt; and
an organic base selected from the group consisting of PPh₃, P(ortho-methylC₆H₄)₃, P(para-methylC₆H₄)₃, ClPPh₂, NPh₃, HNPh₂, P(OMe)₃, P(OPh)₃, Ph₂POPh, AsPh₃, SbPh₃, and BiRₓR′_{y} where x+y is 3 and R, R′ is alkyl, aryl, H, alkenyl, and alkynyl.

2. The buffered ionic liquid of claim 1, wherein M is aluminum, gallium, boron or iron (III).

3. The buffered ionic liquid of claim 1, wherein M is titanium, zirconium, hafnium or aluminum.

4. The buffered ionic liquid of claim 2, wherein M is aluminum, and the compound of the formula RₙMX₃₋ₙ or of the formula RₘM₂X₆₋ₘ is selected from the group consisting of aluminum halide, alkylaluminum dihalide, dialkylaluminum halide, trialkylaluminum, dialuminum trialkyl trihalide; dialkylaluminum alkoxide XAl(OR)₂, X₂Al(OR), Al(OR)₃, RAl(OR)₂, R₂Al(OR); and dialuminum hexahalide.

5. The buffered ionic liquid of claim 4, wherein the compound of the formula RₙMX₃₋ₙ or of the formula RₘM₂X₆₋ₘ is selected from the group consisting of ethyl aluminum dichloride, dialuminum triethyl trichloride, diethyl aluminum ethoxide [(C₂H₅)₂Al(OC₂H₅)], trichloroaluminum (AlCl₃), trichloroaluminum dimer (Al₂Cl₆), diethyl aluminum chloride (Et₂AlCl), and triethyl aluminum (Et₃Al).

6. The buffered ionic liquid of claim 1, wherein the organic halide salt is hydrocarbyl substituted ammonium halide represented by the formula R₄NR₁R₂R₃—Halide, wherein each of R₁, R₂, R₃ and R₄ is H or C₁-C₁₂ alkyl, hydrocarbyl-substituted imidazolium halide; hydrocarbyl-substituted N-containing heterocycles selected from the group consisting of pyridinium, pyrrolidine, piperidine, and the like.

7. The buffered ionic liquid of claim 1, wherein the organic halide salt is selected from the group consisting of 1-alkyl-3-alkyl-imidazolium halides, alkyl pyridinium halides and alkylene pyridinium dihalides.

8. The buffered ionic liquid of claim 1, wherein the organic halide salt is selected from the group consisting of 1-methyl-3-ethyl imidazolium chloride, 1-ethyl-3-butyl imidazolium chloride, 1-methyl-3-butyl imidazolium chloride, 1methyl-3-butyl imidazolium bromide, 1-methyl-3-propyl imidazolium chloride, ethyl pyridinium chloride, ethyl pyridinium bromide, ethylene pyridinium dibromide, ethylene pyridinium dichloride, 4-methylpyridinium chloride, butyl pyridinium chloride and benzyl pyridinium bromide.

9. The buffered ionic liquid of claim 1, wherein the organic base is triphenylphosphine, triphenybismuthine or triphenylamine.

10. The buffered ionic liquid of claim 1, comprising BMIMCl (butylmethyl imidazolium chloride)/AlCl₃:PPh₃.

11. The buffered ionic liquid of claim 1, comprising BMIMCl (butylmethyl imidazolium chloride)/AlCl₃/PPh₃ in a molar ratio of about 0.05-1.5/1-2/0-0.5.

12. The buffered ionic liquid of claim 1, comprising BMIMCl (butylmethyl imidazolium chloride)/AlCl₃/BiPh₃.

13. The buffered ionic liquid of claim 1, comprising BMIMCl (butylmethyl imidazolium chloride)/AlCl₃/BiPh₃ in a molar ratio of about 0.05-1.5/1-2/0-0.5.

14. An olefin dimerization process, comprising:
dimerizing olefins in the presence of a nickel catalyst in an buffered ionic liquid, said buffered ionic liquid comprising a compound of the formula RₙMX₃₋ₙ or of the formula RₘM₂X₆₋ₘ, wherein:
v) M is a metal selected from the group consisting of aluminum, gallium, boron, iron (III), titanium, zirconium and hafnium;
vi) R is C₁-C₆-alkyl,
vii) X is halogen or C₁₋₄-alkoxy;
viii) n is 0, 1 or 2, and m is 1, 2 or 3;
an organic halide salt; and
an organic base selected from the group consisting of: PPh₃, P(ortho-methylC₆H₄)₃, P(para-methylC₆H₄)₃, ClPPh₂, NPh₃, HNPh₂, P(OMe)₃, P(OPh)₃, Ph₂POPh, AsPh₃, SbPh₃, and BiRₓR′_{y} where x+y is 3 and R, R′ is alkyl, aryl, H, alkenyl, and alkynyl;
and wherein said process results in at least 85% dimers.

15. The olefin dimerization process of claim 14, wherein said base is triphenylphospine and said nickel catalyst is

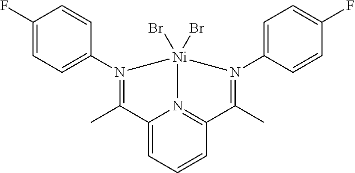

16. The olefin dimerization process of claim 14, wherein said base is triphenylphospine and said catalyst is 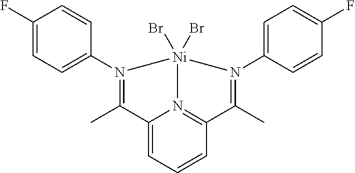
and about 8 equivalents of ethylaluminum dichloride is added per equivalent of catalyst.

17. The olefin dimerization process of claim 14, wherein the buffer is triphenylbismuthine and the catalyst is

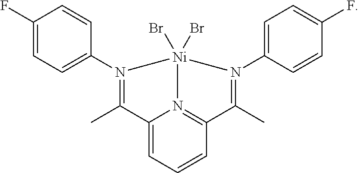

18. The olefin dimerization process of claim 14, wherein said base is triphenylbismuthine, said nickel catalyst is 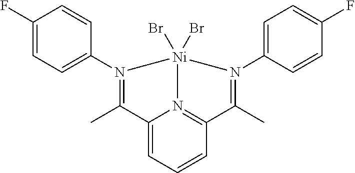
and about 8 equivalents of ethylaluminum dichloride is added per equivalent of catalyst.

19. The olefin dimerization process of claim 14, wherein said dimerizing is carried out under anaerobic conditions.

20. The olefin dimerization process of claim 14. wherein said buffered ionic liquid further comprises a dehydrated silica material on which said buffered ionic liquid is supported.

21. The olefin dimerization process of claim 20, wherein said silica material is treated with ethylaluminum dichloride.

22. The olefin dimerization process of claim 14, wherein said buffered ionic liquid further comprises silica, alumina, titania, zirconia, mixed oxides or mixtures thereof on which said buffered ionic liquid is supported.

23. The olefin dimerization process of claim 20, wherein said buffered ionic liquid is loaded at 80 wt % of said silica support material weight.

24. The olefin dimerization processes of claim 20, wherein said buffered ionic liquid is loaded at 200 wt % of said silica support material weight.

25. The olefin dimerization process of claim 14, further comprising adding at least 0.09 equivalents triphenylbismuthine or diphenyl-Y-bismuthine, wherein Y is a polar or ionic substituent, following the dimerizing step.

26. The olefin dimerization process of claim 25, further comprising adding at least 0.12 equivalents triphenylbismuthine or diphenyl-Y-bismuthine.

27. An olefin dimerization process comprising:
reacting one or more olefins in the presence of a nickel catalyst and a buffered ionic liquid consisting essentially of:
(a) an organic halide salt;
(b) an organic base selected from the group consisting of PPh₃, P(p-XC₆H₄)₃; P(m-XC₆H₄)₃, diphenylphosphinoferrocene, and triphenylphosphino-p-trimethylammonium iodide; and
(c) AlCl₃.
